(19) United States
(12) Patent Application Publication
(10) Pub. No.: US 2015/0087014 A1
(43) Pub. Date: Mar. 26, 2015

(54) METHODS OF SELECTING ALGAE STRAINS FOR PRODUCTIVITY AND ROBUSTNESS

(71) Applicants: Joseph Weissman, Annandale, NJ (US); Robert Nielsen, Lambertville, NJ (US); Roger Charles Prince, Pittstown, NJ (US)

(72) Inventors: Joseph Weissman, Annandale, NJ (US); Robert Nielsen, Lambertville, NJ (US); Roger Charles Prince, Pittstown, NJ (US)

(73) Assignee: ExxonMobil Research and Engineering Company, Annandale, NJ (US)

(21) Appl. No.: 14/035,198

(22) Filed: Sep. 24, 2013

(51) Int. Cl. C12Q 1/04

(52) U.S. Cl. CPC C12Q 1/04
USPC 435/34; 435/288.7

(57) ABSTRACT

Systems and methods are provided to select strains of algal cells for biomass accumulation. Based on synthetic algae sample trajectories, an illumination profile is developed. Strains of algal cells co-cultured in a vessel can then be exposed to the illumination profile under controlled conditions. Properties of algae can be measured and superior strains selected for further cultivation and/or study.

### FIELD OF THE INVENTION

Systems and methods are described for investigating algae properties and selecting improved strains of algae for biomass accumulation.

### BACKGROUND OF THE INVENTION

One potential source of biofuels is to generate molecules from algae that are suitable for making fuels. For example, algae, like plants, can generate lipid molecules. Some lipid molecules have a general structure and molecular weight suitable for making diesel fuel additives such as fatty acid methyl ester (FAME). It is also possible to refine certain algae lipids into conventional fuels or fuel blending stocks including gasoline, diesel, and jet fuel. However, many challenges remain in developing commercial scale production techniques for biofuels based on algae production.

One challenge in further investigating algae based biofuels is identifying algae that will grow effectively in different commercial environments. In a conventional commercial production setting, algae are grown in ponds or other bodies of water that are directly or indirectly impacted by a number of external environmental variables, such as sunlight and ambient temperature. By contrast, typical conventional laboratory settings for studying algae involve little or no exposure to external variables. This reduced exposure to external variables is based on a general desire to screen algae using fixed methods that are repeatable over many test samples. However, conventional methods for introducing this repeatability can lead to laboratory conditions that are not representative of a commercial production environment.

Previous methods for designing photobioreactors have involved using three-dimensional computational modeling of the reaction environment in a photobioreactor. For example, a photobioreactor geometry can be used as a starting point for designing computational fluid dynamic simulations. Based on the photobioreactor geometry, the fluid flow within the photobioreactor can then be modeled to generate trajectories for the movement of algae within the photobioreactor. These simulated trajectories can then be used in combination with a light attenuation model, such as Beer's Law, and a photosynthesis model, to provide simulations that predict algae growth under various conditions. Examples of this type of work include “Simulation of Microalgae Growth in Limiting Light Conditions: Flow Effect” (Pruvost et al., AIChE Journal, Vol. 48. No. 5, p 1109, 2002); “Development of virtual photobioreactor for microalgae culture considering turbulent flow and flashing light effect” (Sato et al., Energy Conservation and Management. Vol. 51, p 1196, 2010); “Scale-down of microalgae cultivations in tubular photobioreactors—A conceptual approach” (Sastre et al., Journal of Biotechnology, Vol. 132, p 127, 2007); and Analyzing and Modeling of Photobioreactors by Combining First Principles of Physiology and Hydrology (Luo et al., Biotechnology and Bioengineering, Vol. 85, p 382, 2004).

PCT International Application Publication WO2006/020177 reports systems and methods for growing algae in a photobioreactor system. The methods include using computational fluid dynamics to calculate trajectories of algae particles in a photobioreactor. Models of photosynthetic behavior for algae are then used to determine desired amounts of light exposure for the algae in the photobioreactor. When algae are introduced into the photobioreactor, the schedule for light exposure is set based on predictions from the photosynthesis model.

### SUMMARY OF THE INVENTION

Models can be used to conceptualize a process and define important parameters that affect the process being modeled. Some parameters that are important to a model of photosynthesis efficiency are optical cross section of a culture of photosynthetic organisms, chlorophyll fraction of the culture, saturating irradiance (Iₛ), photosynthetic maximum (Pₘₐₓ), the initial slope of a photosynthesis vs. irradiance curve, photoinhibition, respiration, and maintenance. However, there is necessarily an uncertainty, and therefore an unreliability, in all measurements of these parameters. In particular, different parameters can affect other parameters in a dynamic fashion. Because certain of these parameters operate with different time constants than others, the dynamic interactions of these parameters impede researchers from measuring each in a way that is useful for modeling the overall photosynthesis system. For example, the decrease ratio of ATP/ADP or NADPH/NADP occurs on the order of seconds, photoinhibition and RuBisCo activation on the order of seconds, activation of antenna-based non-photochemical quenching on the order of tens of seconds (although it might deactivate more slowly), and photo-acclimation occurs on the order of hours. It is not yet clear at what rate processes such as chlororespiration, mitochondrial dark respiration, or maintenance turn on or off. Most estimations of these variables cited in the literature are determined by removing a sample from a larger culture and moving it to another environmental context (a measurement instrument) in which the variable can be measured; these processes introduce an additional level of uncertainty into the estimation of such parameters, because the cells can continue to adjust during the move from culture to instrument. The inventions of the present disclosure use growth simulations to overcome these problems of unreliability. Dilute cultures can simulate growth and mixing of a larger culture setting, whether natural or human-made so as to understand what physiological processes most affect productivity.

In one aspect, the invention provides methods for selecting strains of algal cells for accumulation of biomass, such as proteins, lipids, or polysaccharides. The methods can select strains with improved properties for biomass accumulation, particularly strains whose improved photosynthetic efficiency makes them better able to achieve maximum carbon fixation from a given quantum of light energy.

In said methods, at least two different strains of algal cells are co-cultured in a vessel, for example at least 3 different strains, at least 4 different strains, at least 5 different strains, at least 6 different strains, at least 7 different strains, at least 8 different strains, at least 9 different strains, at least 10 different strains, at least 20 different strains, at least 30 different strains, at least 40 different strains, at least 50 different strains, at least 75 different strains, or at least 100 different strains are co-cultured in a vessel.

In some embodiments the different strains belong to different algal species. In some embodiments, the different strains belong different sub-species of the same species. In some embodiments, the different strains are different mutant varieties of one species. In some embodiments, the different strains are made up of mixtures of different species, sub-species, and mutant varieties of individual species, all cultured together, optionally, in one vessel.

The at least two different strains of algal cells are, during at least a portion of the time that they are cultured together, exposed to an illumination profile comprising light conditions that influence the growth rate of at least one of the strains of algal cells. In certain embodiments, these growth-rate influencing light conditions are photoinhibitory to at least one of the strains of algal cells.

If at least one of the strains of algal cells demonstrates superior photosynthetic efficiency under the growth-rate influencing light conditions, then this strain can be selected for further testing and for use in accumulating biomass. In certain embodiments, the sequence of light-exposure followed by selection can be repeated through one or more additional cycles. In certain embodiment, as this cycle of exposure and selection is repeated, the time of exposure can increase over the progression of cycles. In certain embodiments, this cycle of exposure and selection can be repeated until one strain is dominant in the culture. For example, the dominant strain can comprise at least 50%, at least 60%, at least 70%, at least 80%, at least 90%, at least 91%, at least 92%, at least 93%, at least 94%, at least 95%, at least 96%, at least 97%, at least 98%, at least 99%, or 100% of the cells in the culture. In some embodiments, the dominant strain comes to dominate because it dies less quickly than another strain. In some embodiments, the dominant strain comes to dominate because it multiplies more quickly than another strain.

In certain embodiments, in order to model a desired reference environment, a synthetic trajectory of a particle can be contrived for a hypothetical cell in a reference volume. Contrivance of the synthetic trajectory includes contrivance of at least a plurality of position values in the reference volume. The plurality of position values have an associated set of continuous times and include at least a depth value relative to a light-incident surface of the reference volume. In addition to position values associated with continuous time values, a synthetic trajectory can also include additional information about the microenvironment of the hypothetical cell, such as but not limited to, temperature values, acidity/alkalinity (i.e., pH) values, carbon dioxide concentration values, oxygen concentration values, nitrogen concentration values, and salinity values. Next, an illumination profile can be formed based on the synthetic trajectory. The illumination profile consists of at least a series of illumination intensities corresponding to the plurality of position values and associated times. This can result in an illumination profile corresponding to light intensity that an algae sample would encounter in the reference volume while traveling along the calculated trajectory. The illumination profile can then be used to expose an algae sample to light intensity based on the illumination profile.

A vessel in which at least two strains of algal cells are co-cultured can have a volume less than the reference volume. A product of the optical density for the algae sample and a depth of the sample can be less than about 10.0 cm, is preferably less than about 8.0 cm or less than about 6.0 cm, and in some embodiments may be less than about 4.0 cm, for example, less than about 3 cm, about 2.5 cm, less than about 2 cm, about 1 cm, or less than about 1 cm. During or after exposing the algae sample to the illumination profile, at least one algal property can be measured. The “sample culturing period” is used herein to refer to the time period in which an algae sample is exposed to light intensity according to an illumination profile or a continuous series of illumination profiles, and can be any period of time, but will typically be on the order of days or weeks.

Alternatively or in addition, during the culturing period, sample cultures can be allowed to attain an optical density of at least about 0.5, for example, at least about 1.0 or greater than 1.0. For example, the algae sample may not be maintained at a fixed optical density during the sample culturing period. The transmittance (I/I₀) of light through the algal sample during the sample culturing period can be at least about 20% or at least about 30%, or at least about 40%. The fractional absorption ((I₀−I)/I₀) of the sample culture when it is exposed to an illumination profile can reach levels greater than about 20% or higher than about 30% during the sample culturing period, and can reach levels as high as at least about 65%. For example, the highest fractional absorption value reached by an algae sample during the sample culturing period can be between about 20% and about 30%, or can be between about 30% and about 65%, for example between about 30% and about 60%, between about 40% and about 60%, or between about 40% and about 55%. In certain embodiments, the at least two strains of algal cells can be cultured in a turbidostat, such that a substantially constant optical density is maintained during part or all of the sample culturing period.

Further, in some embodiments, the illumination profile can be modified during the sample culturing period based on optical density measurements made during the sample culturing period. For example, in methods where the sample culture is allowed to increase in optical density, the illumination profile can be modified to incorporate attenuation calculations based on updated optical density values. For example, the optical density of an algae sample can be measured one or more times during the sample culturing period to obtain updated optical densities that are used to modify the illumination profile to which the algae samples are exposed as the sample culturing period continues.

The methods above can also allow for comparison of properties of multiple algae samples. For example, two algae samples can be exposed to an illumination profile. The algae samples can be exposed to the illumination profile in different vessels, or the same vessel can be used to consecutively expose the algae samples. One or more properties of the algae samples can be measured, such as, for example, productivity (biomass accumulation), a photosynthetic property, gene expression, a biochemical property, or biomolecule production. In certain embodiments an environmental or physiological parameter of the algal cells or culture can be measured, such as temperature, pH, carbon concentration, oxygen concentration, and cell fluorescence. In certain embodiments, properties related to photosynthetic efficiency can be measured, such as respiration, specific growth rate, pigment concentration (e.g., chlorophyll concentration), oxygen evolution, carbon fixation, and electrical potential across membranes (e.g., chloroplast membranes). As appropriate, these measurements can be made by sampling off-gas from the culture. In certain embodiments properties of the algal cells can be measured following light exposure. One or more environmental conditions can optionally be varied while exposing the samples to the illumination profile.

In another aspect, systems are also provided for performing the culturing, exposure, and selection method steps described herein. Such systems can allow for exposure of an algae sample to an illumination profile under controlled conditions. A system can typically include a selection vessel having a depth of about 10 cm or less, for example, about 4 cm or less, about 2 cm or less, or 1 cm or less, and a cross-sectional area, the depth and the cross-sectional area corresponding to a vessel volume of less than about 5 liters, for example, less than or equal to about 1 L or less than or equal to 500 mL. The selection vessel is connected to a culture vessel, which preferably has a larger volume than the selection vessel. In some embodiments, the culture vessel is hydraulically connected to the selection vessel. In certain embodiments, this hydraulic connection comprises a flow cytometer. The selection vessel and the culture vessel can each be used in conjunction with a plurality of light sources positioned so that emitted light is incident on a vessel surface having an area corresponding to the cross-sectional area. Light from the plurality of light sources can be focused or otherwise directed toward the cross-sectional area using a plurality of lenses positioned to increase the percentage of emitted light that is incident on the vessel surface. It is noted that the dimension corresponding to the depth of the vessel does not need to correspond to the direction of gravitational pull. Instead, the vessel can be oriented in any manner that is convenient for allowing the plurality of light sources to be incident on the cross-sectional area. A system can further typically comprise a memory for storing at least a portion of an illumination profile and a processor. The processor can control at least one power source for the plurality of light sources based on at least a portion of an illumination profile. This can allow the illumination profile to be replicated by the plurality of light sources. Preferably, the plurality of light sources and the plurality of lenses can be positioned to be capable of delivering at least about 1000 μE/m²/sec PAR of illumination to the cross-sectional area of the culture vessel surface and at least about 10000 μE/m²/sec PAR of illumination to the cross-sectional area of the selection vessel surface.

### BRIEF DESCRIPTION OF THE DRAWINGS

FIG. 1A shows irradiance as a function of depth of penetration into solutions, with three different extinction coefficients. FIG. 1B shows photosynthesis as a function of irradiance. Saturating irradiance (Iₛ) is indicated as the level of irradiance where the slope of the initial cure intersects with the photosynthesis maximum value.

FIG. 2A shows a plot of the Bush efficiency (measured in percentage terms) as a function of saturating irradiance over initial irradiance. FIG. 2B illustrates average Bush efficiencies over the day for the indicated saturating irradiances.

FIG. 3A shows cultures in growth vessels at low light intensity (50 μE/m²/sec). FIG. 3B shows cultures in growth vessels at high light intensity (900 μE/m²/sec).

FIG. 4 shows the specific growth rate (p) of a culture of Tetraselmis sp. 2286 as a function of irradiance (I).

FIG. 5 shows the results of a light shift on the specific growth rate of Tetraselmis cultures. Cultures of Tetraselmis sp. 2286 cells were acclimated over extended time periods to growth at the culture conditions indicated to the right of the chart. Following acclimation, the cultures were shifted to the light conditions indicated on the x-axis of the chart, and specific growth rates were measured over a three hour growth period.

FIG. 6 shows the natural log of irradiance over a period of time in a culture medium with an extinction coefficient of 200 m⁻¹. This pattern of light intensities constitutes a “stair case” illumination profile.

FIG. 7A shows simulated productivity achieved with algal cultures exposed to different light intensities, at different fluid transparencies, applied in different patterns. FIG. 7B shows simulated specific growth rates of certain of the cultures shown in FIG. 7A.

FIG. 8 shows a series of algal culture ponds. The surface of the pond in 8A is 67% covered in shade. This is achieved by placing a series of 15 cm wide slats across the pond surface, with 7.5 cm gaps between each slat. The surface in 8B is only 45% covered in shade, which was achieved by placing a series of 15 cm wide slats across the surface, with 15 cm gaps between each slat. The surface in 8C is 48% covered in shade, which is achieved by placing a series of 7.5 cm wide slats across the surface, with a 7.5 cm gap between each slat. The surface of the control pond in 8D is completely unshaded. The photosynthetic efficiencies achieved from cultures in these various ponds are summarized in Table 2.

### DETAILED DESCRIPTION OF THE EMBODIMENTS

### Definitions

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

To facilitate an understanding of the present invention, a number of terms and phrases are defined below.

As used in the present disclosure and claims, the singular forms “a,” “an,” and “the” include plural forms unless the context clearly dictates otherwise.

Wherever embodiments are described herein with the language “comprising,” otherwise analogous embodiments described in terms of “consisting of” and/or “consisting essentially of” are also provided.

The term “and/or” as used in a phrase such as “A and/or B” herein is intended to include “A and B”, “A or B”, “A”, and “B”.

The terms, “cells”, “cell cultures”, “cell line”, “recombinant host cells”, “recipient cells” and “host cells” as used herein, include the primary subject cells and any progeny thereof, without regard to the number of transfers. It should be understood that not all progeny are exactly identical to the parental cell (due to deliberate or inadvertent mutations or differences in environment); however, such altered progeny are included in these terms, so long as the progeny retain the same functionality as that of the originally transformed cell.

The terms “naturally-occurring” and “wild-type” (WT) refer to a form found in nature. For example, a naturally occurring or wild-type nucleic acid molecule, nucleotide sequence, or protein may be present in, and isolated from, a natural source, and is not intentionally modified by human manipulation.

The term “photosynthetic organism” as used herein is any prokaryotic or eukaryotic organism that can perform photosynthesis. Photosynthetic organisms include higher plants (i.e., vascular plants), bryophytes, algae, and photosynthetic bacteria. The term “algae” includes, but is not limited to, a species of Bacillariophyceae (diatoms), Bolidomonas, Chlorophyceae (green algae), Chrysophyceae (golden algae), Carophyceae, Cyanophyceae (cyanobacteria), Eustigmatophyceae (pico-plankton), Glaucocystophytes, Pelagophytes, Phaeophyceae (brown algae), Prasinophyceae (pico-plankton), Raphidophytes, Rhodophyceae (red algae), Synurophyceae and Xanthophyceae (yellow-green algae). The term “algae” includes microalgae. The term “microalgae” as used herein refers to microscopic, single-celled algae species including, but not limited to, eukaryotic single-celled algae of the Bacillariophyceae, Chlorophyceae, Prasinophyceae, Trebouxiophyceae, and Eustigmatophyceae classes. The term “photosynthetic bacteria” includes, but is not limited to, cyanobacteria, green sulfur bacteria, purple sulfur bacteria, purple non-sulfur bacteria, and green non-sulfur bacteria.

The term “strain” as used herein refers to a genetic variant or subtype of a microorganism (e.g., bacterium or protist). The phrase “at least two strains” can embrace two or more different species, two or more different subspecies within a single species, two or more different mutant members of the same species or the same subspecies, and/or any combination of these categories. The “dominant strain” is a strain, the cells of which constitute a plurality of the cells in a mixed culture; therefore, when three or more strains are cultured together, a single strain can be the “dominant strain” even without attaining a numerical majority of the cells within the culture. For example, if there are three cultures being co-cultured (A, B, & C) that begin in equal proportions (1:1:1), but after a number of rounds of selection, A=40% of the cells, B=30%, and C=30%, then A would the “dominant” strain, even though there are still more “non-A” cells in the culture than A cells. In other words, depending on the context, including the number of strains present in a mixed culture, a strain comprising 10%, 20%, 30%, 40% 50%, 60% 70%, 75%, 80%, 85%, 90%, 95%, 96%, 97%, 98%, 99%, or even 100% of the cells in the culture can be designated the “dominant strain.”

The term “biomass” as used herein, refers in general to organic matter produced by a biological cell. The renewable biological resource can include microbial materials (including algal materials), fungal materials, plant materials, animal materials, and/or materials produced biologically. “Biomass” should be understood to include proteins, lipids, and polysaccharides, whether retained within a biological cell or excreted from a biological cell, in addition to other molecules synthesized by a biological cell. As used herein, biomass is “excreted” from a cell whenever it is found outside of a cell, regardless of whether or not the egress of biomass occurred as a result of cellular activity.

The term “photosynthetic efficiency,” as used herein, refers to a measure of the ability of a photosynthetic organism to convert energy from actinic light into biomass. As photosynthetic efficiency increases, a larger percentage of the actinic light energy that a photosynthetic organism encounters is converted into biomass. In certain circumstances it is useful to speak of“relative photosynthetic efficiency.” In these circumstances, a particular photosynthetic organism is set as a reference standard. The photosynthetic efficiency of another photosynthetic organism is described in relation to the reference standard photosynthetic organism (e.g., 50% as efficient, 200% as efficient, etc.). A “photosynthetic efficiency characteristic” is a feature or element of a photosynthetic cell that affects or reflects the photosynthetic efficiency that a cell achieves under on or more circumstances. Exemplary photosynthetic efficiency characteristics include specific growth rate, pigment content (including chlorophyll content), oxygen evolution, carbon fixation, and tolerance of biomembranes (including chloroplastic membranes) to electrical potential.

“Photosaturating light conditions” are light conditions in which the cell is inundated with illumination, such that the volume of light energy entering the cell exceed the cell's capacity to convert light energy into biomass. Therefore, photosaturating light conditions depress photosynthetic efficiency, because portions of energy entering the cell are simply lost for incapacity to process them. Photosaturating light conditions can induce the activation of a cells nonphotochemical quenching mechanisms, which result in light energy being dissipated as heat instead of being fixed into biomass. Photosaturating conditions that so greatly exceed the cell's capacities that the excess energy damages the cell's photosynthetic machinery are referred to as “photoinhibitory light conditions.”

As used herein “pond” means any open body of water, whether naturally-occurring or man-made, including ponds, canals, trenches, lagoons, channels, or raceways. The open pond can have a depth of from about 3 cm to about 500 cm, and will typically have a depth of from about 5 cm to about 100 cm, such as from about 8 cm to about 50 cm, or from about 10 cm to about 40 cm.

The terms “artificial light” or “artificial source of light” are used herein to refer to any type of light different from sunlight. Thus, artificial light can include incandescent sources, fluorescent sources, light emitting diode sources, or any other convenient source for generating light.

The movement of a particle through a reference volume, such as a pond, is a “particle trajectory” for purposes of the present application. A particle trajectory comprises at least a position value relative to a light-incident surface of a reference volume and a time value corresponding to the position value. Some particle trajectories occur with low probability or not at all in a reference volume as a result of natural fluid dynamics. As used herein, “synthetic trajectory” refers to a particle trajectory that is unlikely or impossible to occur as a result of natural fluid dynamics. For example, while it is theoretically possible for a particle never to move at all, when actual particles are observed in fluids, completely stationary trajectories are not frequently observed. Certain trajectories are unlikely because they are not possible within the physical constraints of the natural universe; such an impossible trajectory is designated an “unreal trajectory” (i.e., not naturally occurring) for purposes of the present disclosure, and should be understood as a subset of the category “synthetic trajectories.” An example of an unreal trajectory is a cell moving through a liquid at a rate so rapid that the forces that the cell experiences, such as shear forces and collisions with other particles, would destroy the cell. Certain unreal trajectories involve a plurality of discontinuous position values associated with a corresponding series of continuous time values, as if the particle were moving from a depth of, for example, one centimeter to a depth of, for example, three centimeters, without ever passing through a depth of two centimeters; a trajectory according to this subset of unreal trajectories is a “discontinuous trajectory” for purposes of the present disclosure. One skilled in the art will appreciate that the examples of “synthetic trajectories” and “unreal trajectories” presented above are non-limiting, and that many other possible trajectories belong to each category.

When used in reference to a trait or characteristic (e.g., specific growth rate, rate of oxygen evolution, etc.), the words “diminished” and “attenuated” are interchangeable and mean “reduced in amount, degree, intensity, or strength.” Similarly, when used in reference to a trait or characteristic, the words “enhanced” or “increased” are interchangeable and mean “having a greater amount, intensity, degree, or strength.” In other words, “attenuated” and “diminished” are antonyms of “enhanced” and “increased.” An algal strain X is said to have an “enhanced” trait when the degree or intensity of the trait observed in strain X is greater than is observed in strain Y. The same applies, mutatis mutandis, to “increased,” “attenuated,” and “diminished.”

When used herein to describe a step-wise change, “discrete” means “having consecutive values that are not infinitesimally close, so that its analysis requires summation rather than integration.”

### Overview

Systems and methods are provided for growing algae under selection conditions that would be unlikely or impossible to occur in a natural setting. In certain embodiments, the growth conditions are selected based on a cellular model, such as a mathematical model, of photosynthesis. Individual parameters of a cellular model of photosynthesis can be tested by contriving a synthetic trajectories of a particle that would result if the parameter of interest were operating at a rate or degree that would be unlikely or impossible to achieve with organisms in a natural setting. In combination with a light attenuation model, and preferably in combination with a model of light incidence, contrived synthetic trajectories can be used to form light exposure or illumination profiles for algae. Optionally, the model can be supplemented with model information and/or measured information regarding any variable that may change in space or time during cultivation in a reference environment. Examples of a supplemental model and/or measured information include but are not limited to the optical density of the culture in the test vessel, the temperature of the reference environment, the carbon-dioxide content and/or pH of the reference environment, the oxygen content of the reference environment, the nitrogen content of the reference environment, and the salinity of the reference environment. The algae can then be placed in a tank or vessel and exposed to light based on the illumination profile as well as other model environmental conditions as desired by the investigator. Alternatively, conditions such as temperature can be fixed, such as by placing the vessel in a temperature bath. Alternatively, one or more variables can be set initially and then allowed to vary based on the ongoing growth reactions in the vessel. The inventive systems and methods allow for growth and/or maintenance of algae in a test vessel under conditions that may, for at least certain variables or parameters, be selected to model a larger scale reference environment, such as a photobioreactor, a raceway pond, or any other commercial production environment. When two or more strains of algal cells are co-cultured in these conditions, the illumination profile used may favor the growth of one strain over the other(s), and in this way strains with superior characteristics can be selected. Additionally, a strain selected in this way can be continuously cultured under the illumination profile conditions used to select the strain, so as to maintain the strain's superior characteristics.

### Methods of Strain Selection

The systems and methods of the present invention can be used to test cell models of photosynthesis, and to use information obtained from cell models of photosynthesis to select strains of algal cells with enhanced photosynthetic efficiency. In certain embodiments, circumstances are contrived in which the ability of a microorganism to convert actinic light energy into biomass is the rate limiting factor for cell replication. In other words, the illumination profile, and therefore the light conditions, used in the methods of the present invention can influence the growth rate of the strains of algal cells in ways that select for strains better adapted for biomass accumulation in industrial or laboratory settings. For example, strains can be cultured in a medium lacking a reduced carbon source, such that the only source of metabolic energy comes from photosynthesis. When strains of algal cells are cultured in such a medium under conditions that are photoinhibitory for some cells, the cells for which these conditions are not photoinhibitory will progressively come to predominate in a mixed culture over less efficient strains. In certain embodiments, circumstances are contrived in which cells that are not sufficiently efficient at photosynthesis will die, such that the more efficient cells will progressively come to predominate in a mixed culture. In certain embodiments, the photosynthetic efficiency of individual algal strains can be tested and strains that show superior efficiency can be selected for subsequent culture.

Based on an illumination profile and one or more corresponding condition profiles, various strains of algal cells can be co-cultured under conditions contrived to select for those cells that display the greatest photosynthetic efficiency. The trajectories can be used to determine an illumination profile. Additionally, a corresponding temperature profile can be determined. If desired, a corresponding pH profile, O₂ profile, and/or a profile for other controlled variables can be developed.

Using the illumination profile, various strains of algal cells with different physiological properties can be co-cultured in a growth vessel under circumstances that will select for one strain or another. The rate at which this selection can occur will depend on the fitness traits of the strains being selected and the strenuousness of the selection conditions. For example, in appropriate nutrient-rich conditions, Tetraselmis algae in logarithmic phase divide every 20 hours (see, Gopinathan (1986) Indian J. Fisheries 33(4):450-56). Therefore, if two competing strains begin at equal proportion in a co-culture, and one strain of Tetraselmis cells (strain A) has a 5% fitness advantage over another strain (strain B) in the context of a particular trajectory, it requires only 17.5 days under the trajectory conditions before A will be twice as prevalent in the culture as B. Of course, the period of time necessary to achieve this degree of enrichment of the more fit strain will be less if the fitness advantage is starker (such as 10%, 15%, 25%, etc.), and the time will be longer if the fitness advantage is smaller (such as 4%, 1%, 0.5%, etc.). The period of time necessary to enrich for the more fit strain will also vary according to the proportion of cells from each strain at the start of the selection conditions. That is to say, if, unlike in the example above, the strains do not start in equal proportion, it will require more or less time for the more fit strain to emerge as dominant.

In certain embodiments, the strains in the co-culture can be subjected to multiple rounds of selection. For example, strains of algal cells in co-culture can be subjected to 2, 3, 4, 5, 6, 7, 8, 9, 10, 20, 30, 40, 50, 60, 70, 80, 90, or even 100 or more rounds of selection. In this way, with each round of selection the culture will become more enriched for the strain with the desired trait. If desired, the selective pressure can be increased, gradually or acutely, over repeated rounds of selection, for example by increasing the length of time during which cells are exposed to a selective pressure. In certain embodiments, one particular strain, the “dominant” strain, will come to predominate numerically in the culture with time. In certain embodiments, it is desirable to isolate pure or substantially pure cultures of this dominant strain by means of techniques, such as limited dilution and soft-agar culture, well known to those of skill in the art.

In certain embodiments, it is desirable to measure cell responses to illumination conditions. Cells can respond to light stimulus in a variety of ways, including by responses that manifest as changes in parameters such as temperature, pH, carbon concentration, and/or oxygen concentration of the cell (physiological change) or its micro-environment (environmental change). Cells can also respond by changing their pigment compositions (including their chlorophyll composition), which can result in a change in cellular fluorescence. Measurements of these changes can be made in many ways. Where the cell emits a product in response illumination conditions, such as a starch, a lipid, or a volatile organic compound, it may be possible to sample this effluence by removing samples of culture medium or off-gas from the culture. In other embodiments, it may be useful to inspect samples of the cells visually under a microscope, or via flow cytometry. In certain embodiments, when cells are examined by flow cytometry, it is possible to remove cells that do not display desired characteristics, so as to enrich the remaining growth culture for cells possessing desired traits.

### Synthetic Trajectories and Formation of Illumination Profiles

As an initial step, in any of the systems and methods, particle trajectories and corresponding illumination profiles must be formed. In certain embodiments, the particle trajectory will be a synthetic trajectory. In certain embodiments, computational fluid dynamics (CFD) calculations can be used to predict a trajectory or set of trajectories for an algal cell moving through a growth environment. Various computational fluid dynamics calculation programs are available, such as Fluent from Ansys, Inc. of Canonsburg, Pa.

The geometry of the growth environment can be created within the computational fluid dynamics program. The volume in the geometry can then be divided into a mesh or grid of volume elements to allow for calculation of a flow within the geometry of the growth environment. A solution can then be calculated for the flow within the geometry, or a flow pattern can be calculated to incorporate an energy source, such as a paddle wheel for a raceway pond geometry. In some embodiments, a steady state solution for the flow can be sufficient even though the corresponding system being modeled includes a discontinuous energy source.

After developing a flow solution for the geometry, test particles representing an algae cell can be introduced into the modeled flow pattern. The trajectories or traces of test particles can be tracked and recorded as the test particles travel through the geometry. For example, if the reference geometry being modeled represents a raceway pond, particle trajectories or traces can be modeled that correspond to one circuit for a particle around the pond. Thus, each individual trajectory can correspond roughly to a period of time. The period of time can be a few minutes, about 0.5 hours, about an hour, about 2.5 hours, about 1 day, about 3 days, about 5 days, about 7 days, or any other period of time that is desired. A particle trace or trajectory can represent a series of discrete location values, or the trajectory can be a continuous position function. A particle trajectory includes at least a depth value for a particle as a function of time. Typically, a particle trajectory can also include lateral coordinates indicating a location within a reference geometry. However, the coordinates in a particle trajectory do not necessarily have to be expressed as Cartesian coordinates. For example, in the case of circular or annular reference geometries, it may be more convenient to use coordinates corresponding to a depth, a radial position, and an angular position. It is noted that, even if a coordinate system does not explicitly have a coordinate expressing a depth relative to a surface in the geometry that is exposed to incident illumination, it is sufficient for purposes of this invention that the depth relative to a surface in the geometry can be derived from the position coordinates. Thus, any set of information for a trajectory that can provide, directly or indirectly, a depth coordinate can be considered equivalent to explicitly having a depth coordinate for the trajectory.

For an existing reference system, in any of the systems and methods, an alternative to computational modeling can be to measure fluid velocities in the reference system. The measured velocities can be used to develop a fluid flow field model for the reference system. Using methods similar to a CFD calculation, the measured fluid flow field can be used to generate random particle trajectories or traces. In one embodiment, a more accurate computational model can be created by tuning model variables, and validating predicted results, with measured fluid velocities in the reference system.

The process for modeling a trajectory or trace can be repeated any convenient number of times in order to generate a library of trajectories, which can be on the order of at least about 100 trajectories, for example at least about 1,000 or at least about 10,000. The trajectories from the library can then be selected for use in determining illumination exposure conditions for algae. For any given characteristic of the trajectory (e.g., particle time spent at the surface, total volume of photons encountered, etc.), trajectories can be fit into a bell curve distribution. Statistical outliers (the left and right “tails” of the curve) would consist of trajectories with a low probability of occurring in a natural system subject to physical laws. These low probability trajectories are rare because they represent occasions when a particular variable in reference system is given an extreme value. These low probability trajectories can be used to test the effect of the variable on the photosynthetic system. For example, trajectories found at the right or left tail of the distribution and occupying less than 10%, for example less than 5%, less than 1%, less than 0.1%, or less than 0.01% of the area under the curve can be selected for testing a variable of interest. A plurality of trajectories can be chained together to form a longer synthetic trajectory corresponding to the desired time period.

In certain embodiments, the synthetic trajectory can be a trajectory that cannot occur in natural settings, i.e. an unreal trajectory. For example, the systems and methods of the present invention can be used to simulate the light conditions that an algal cell would encounter while moving through a fluid at a velocity that would destroy the cell. By way of additional or alternative example, the systems and methods of the present invention can be used to investigate the effect of light conditions that do not occur in nature (e.g., illumination more intense than natural sunlight, or illumination sustained for extended periods that do not occur on Earth, such as four months of continuous, uninterrupted illumination). By way of further additional or alternative examples, the systems and methods of the present invention can be used to simulate abrupt transitions between light intensities that a particle moving in a natural reference volume would not encounter, such an instantaneous movement from high light to darkness, with no transition in between. The skilled reader will understand that the examples given in this paragraph are illustrative, and should not be understood to limit the description of these unreal trajectories.

Based on a synthetic trajectory of a given length of time, an illumination profile can be created that corresponds to the trajectory. An illumination profile represents the actual light an algae cell should receive if the algae cell were to follow the synthetic trajectory in the reference growth system. e.g., an open, closed, or hybrid pond or bioreactor. The illumination profile can take into account several factors. First, the characteristics of the source of illumination (e.g., intensity, duration, and wavelength) should be considered. In some embodiments, the goal is to model a pond or other body of water that is exposed to sunlight. In such embodiments, the sunlight can be directly incident on the surface of the body of water, or the sunlight can be attenuated prior to reaching the water. This could represent, for example, a small pond located within a greenhouse type structure. Alternatively, the illumination profile can model an artificial source of light, such as the artificial light sources used in some photobioreactors. Still another option can be to model a structure surrounding a body of water that allows sunlight to enter, but that can also include artificial sources of light.

Based on the source of light, the illumination incident on the surface of the body of water in the reference geometry can be determined during the time period for the trajectory. As an example, consider a trajectory representing algae movement through a pond during daylight hours on a day having 12 hours of daylight. During the course of the day, varying amounts of sunlight will be incident on the surface of the pond. The incident sunlight on the pond can be represented in any convenient manner. One option can be to determine a maximum amount of light that will be incident on the body of water, and then express the incident light at other times as a fraction of the maximum incident light.

In an example involving a pond exposed to sunlight, an illustrative maximum sunlight intensity on a pond surface is about 2000 μE/m²s of photosynthetically active radiance (PAR). Photosynthetically active radiance refers to light intensity between about 400 nm and about 700 nm, which corresponds to light intensity that participates in typical photosynthetic processes. This number could vary depending on a variety of factors, such as the time of year, a latitude selected for the model pond, or an expected amount of cloud cover. In alternative embodiments, still other factors could be accounted for in determining the amount of light incident on the surface of a body of water. Accordingly, in various embodiments, the maximum sunlight intensity can be selected to be at least about 400 μE/m²/s PAR, for example at least about 800 μE/m² is PAR, at least about 1000 μE/m²/s PAR, or at least about 1500 μE/m²/s PAR. Additionally or alternatively, the maximum sunlight intensity can be selected to be about 2400 μE/m²/s PAR or less, for example about 2000 μE/m²/s PAR or less or about 1500 μE/m²/s PAR or less. Based on the selected maximum, the illumination incident on the pond surface during the course of the day can be calculated based on the angle of the sun. The incident illumination can optionally be calculated as a continuous function, to reflect the continuous nature of the change in sunlight intensity over the course of a day. Alternatively, the incident illumination can be calculated in a discreet manner, such as assigning an average illumination intensity for each 6 second period, each 10 second period, each 1 minute period, each 10 minute period, each 15 minute period, and/or for any other convenient period of time.

The above example relates to determining intensity for a pond exposed to sunlight. In other embodiments, at least a portion of the light incident on a body of water can be artificial light. In such embodiments, the amount of light incident on the body of water can be calculated based on factors such as the output illumination intensity of the light sources and the percentage of the source illumination that contacts the surface of the body of water.

Based on the amount of light incident on the surface of a body of water, an illumination profile for algae can be determined by using the trajectory for the algae to calculate an amount of light attenuation for the incident light. As algae move through a body of water, the location of the algae relative to the surface of the body of water will typically vary. Due to light attenuation, the amount of illumination the algae are actually exposed to will typically vary. When the algae are closer to a surface where light is incident, the algae will be exposed to a greater amount of incident light. When the algae are at a greater distance from a surface where light is incident, the amount of light intensity reaching the algae will be reduced.

The attenuation of light as it passes through a body of water can be expressed as an optical density for the algae-containing water. One way of expressing optical density can be as a percentage of light attenuation per centimeter of depth for the water. Optical density through the sample, or absorbance (A_{λ}), is inversely related to the transmittance of light through the sample: A_{λ}=log₁₀(I₀/I), where I₀ is the incident light intensity and I is the intensity of the light after it passes through the sample, and transmittance=(I/I₀), often expressed as a percentage.

In some embodiments of the invention, algal cells, including cultures of one strain, co-cultures of two strains, co-cultures of three strains, co-cultures of four strains, or co-cultures of five or more strains can be cultured in a vessel of dimensions and materials sufficient to ensure that no more than 40%, for example no more than 30%, no more than 25%, no more than 20%, no more than 15%, no more than 10%, or no more than 5% of light intensity is lost across the optical depth of the vessel. In other embodiments, the density of the culture is sufficient to result in a loss of more than 20%, for example more than 25%, more than 30%, more than 35%, 1 to or more than 40% of light intensity across the optical depth of the vessel.

When determining an illumination profile, the optical density of the algae-containing water can be set to any convenient value. Optical density (OD) is often used as a correlate of algal cell concentrations, where an optical density for determining algal cell concentration can be determined at wavelength outside the PAR range, for example, at 730 nm or 750 nm, so that differences in the amount of photosynthetic pigments of the cells (which can vary, for example, with growth conditions) do not improperly influence estimates of cell concentration. Depending on the type of algae, an optical density of 1.0 (for example, at 730 nm) can correspond to a biomass concentration in water of from about 0.3 g/liter to about 0.5 g/liter. Typical maximum optical density values for a raceway containing algae in an aqueous media range from about 0.5 to about 0.75, but in some instances may be higher, for example, optical densities in raceway ponds may be greater than 1.0, depending on factors such as algae concentration and absorptivity, but any optical density value can be selected for a simulator culture that models a desired growth environment.

The optical density for a body of algae-containing water can correspond to an absorption coefficient in Beer's Law. As described above, optical density corresponds to an amount of absorption per centimeter (or another convenient length unit). Based on an optical density (or absorption coefficient) a and a path length L, Beer's law can be written as:

αL=−log₁₀(I_{L}/I₀)=εLc

where αL is the absorbance (A_{λ}) of the culture at a particular wavelength, I₀ represents the intensity of light incident at the surface, and I_{L} represents the intensity of light at distance L from the surface. If additional information is available regarding the types of algae present in the water, it may be possible to calculate the absorption coefficient or optical density α at a wavelength of the incident light (i.e., a PAR wavelength) based on the concentration c of algae in the water and the molar absorptivity or extinction coefficient, ε, of the algae at the incident light wavelength. The extinction coefficient of an algal strain can be determined by measuring light intensity at various depths of an algal culture, such as a pond culture, where the concentration of the algae and the intensity of incident light are known. The extinction coefficient of a strain (measured at a PAR wavelength) may vary over time in culture, as the strain can adapt to culture conditions by increasing or decreasing pigments that absorb PAR. Extinction coefficients may therefore optionally be determined for an algal strain at various times during culturing, for example, extinction coefficients can be measured daily for a period of several days, a week, or several weeks, to provide extinction coefficients for the strain at various stages of culturing of the strain. One or more extinction coefficients obtained for an algal strain can be used to form the illumination profile, where the illumination profile can, as desired, vary over the culture period at least partly to take into account the variation in the extinction coefficient over time in a culture. Extinction coefficients used to form an illumination profile for a given algal strain can also or alternatively be extinction coefficients obtained from one or more culture measurements of a similar algal strain. For example, illumination profiles can be formed for a given strain based on one or more extinction coefficients calculated for one or more strains of the same algal taxon, such as the same algal family, order, class, and/or genus.

The extinction coefficient (K) of a culture can be calculated with the following formulae: K=(m²/g_{chla})(g_{chla}/g_{C})(g_{C}/m³), where m²/g_{chla} is the average optical cross section of the dilute suspension that is used in the simulation, g_{chla}/g_{C} is the fraction of the cell carbon that is chlorophyll, and g_{C}/m³ is the particulate organic carbon density (C) of the dense culture being simulated. The average optical cross section of the dilute suspension can be measured by taking the suspension, measuring the absorption spectrum in an integrating sphere spectrophotometer, and calculating an average optical cross section, using the absorption spectrum and the emission spectrum of the lights. C is half of X, where X is the biomass density of the dense culture as simulated, measured in grams of ash-free dry mass per liter of culture. An exemplary dense culture has a K value ˜100.

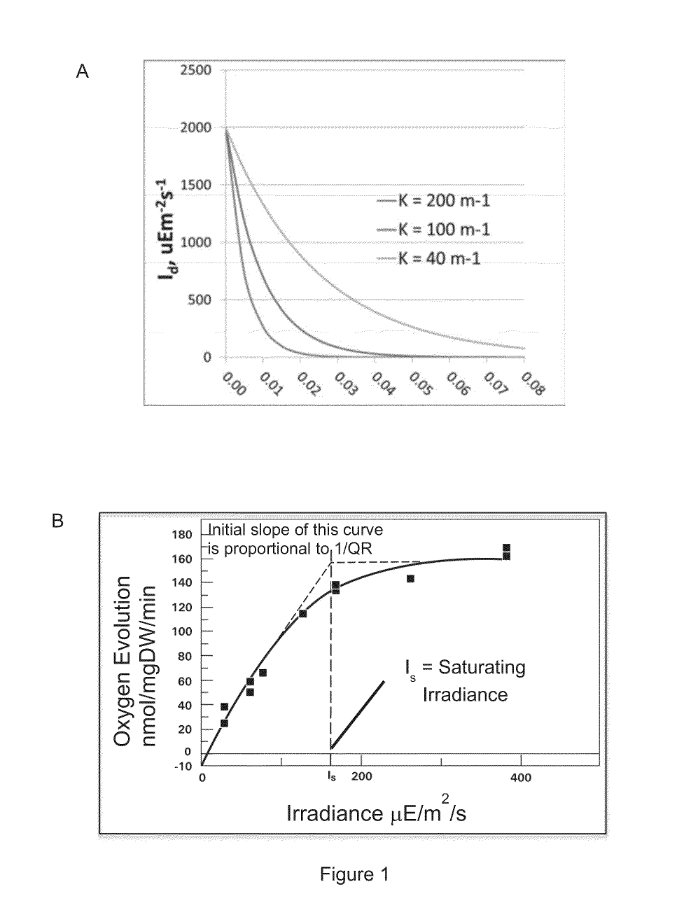

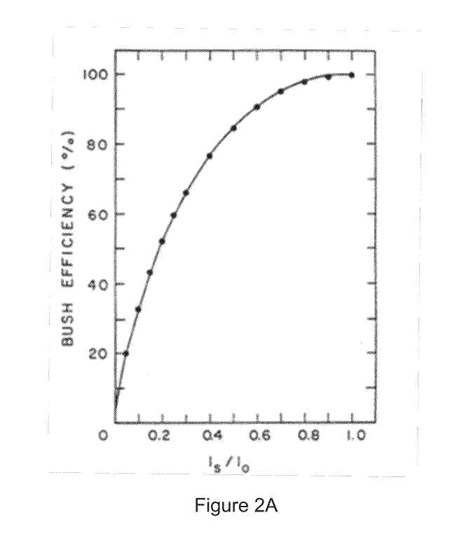

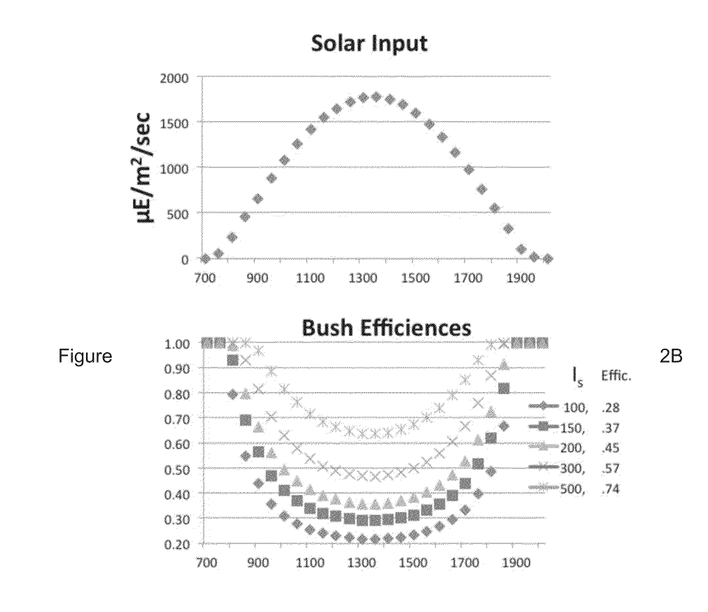

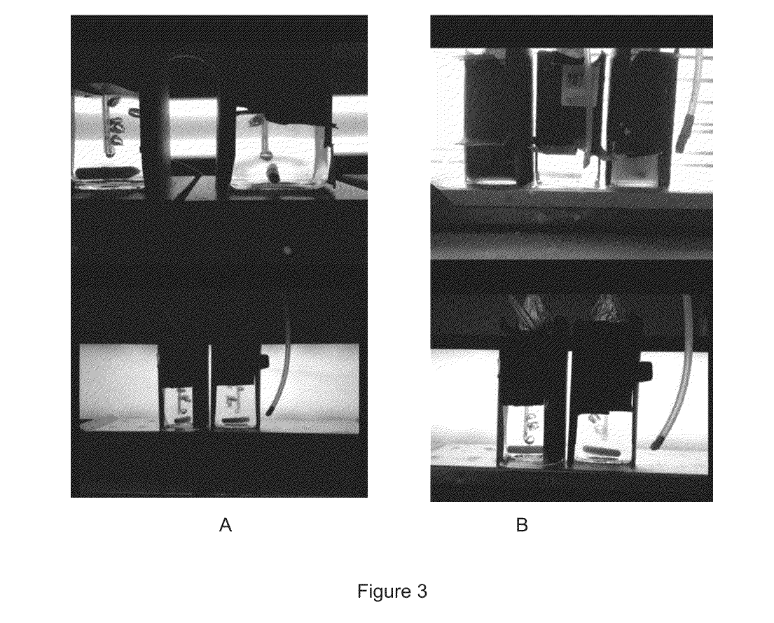

“Productivity” in this context is μX, where μ is the specific growth, calculated according to the formula Ln(biomass (t₂)/biomass (t₁))/(t₂−t₁), where t₂ is a time point arbitrarily later than t₁. When the ash free dry weight of a dense culture is measured in g/L, one can multiply by the volume (liters) of culture per square meter of illuminated surface to get grams of ash free dry weight/m²/day as the productivity of the simulated dense culture. Productivity can also be modeled as C·QE·I₀·Bush efficiency−mX, where QE is the quantum efficiency, I₀ is initial irradiance, m is the specific maintenance, and X is biomass (see, FIG. 1B). Bush efficiency is given by the formula Iₛ/I₀(1−Ln(Iₛ/I₀)), where Iₛ is the saturating irradiance (see, FIG. 2A).

For convenience, in forming an illumination profile, a single optical density value can be used for all incident wavelengths of light. Additionally or alternatively, the amount of light attenuation could be refined by using an optical density function that provided a wavelength dependent value for optical density. In some embodiments, the optical density value used to calculate attenuation can change over time, to reflect or model changes in the density of algae in the body of water. For an existing body of water or reference geometry, the optical density under various conditions can be measured. Alternatively, for any reference geometry, the optical density can be selected to have a desired value, such as an optical density corresponding to a desired concentration of algae in the body of water.

Based on the optical density for the algae-containing water, the amount of light intensity reaching algae at a given point in a trajectory can be calculated in order to provide an illumination profile for the algae. The trajectory can provide the depth of the algae in the water. The amount of light incident on the surface of the water can be determined or modeled based on the light source(s). The optical density can provide the information needed to determine the intensity of light reaching the algae based on the intensity of light at the water surface. Based on Beer's law, these values can generate the light incident on the algae at each point in the trajectory. This series of incident light values corresponds to an illumination profile (or illumination history) for the algae.

In using simulator cultures to model a reference algal culture, in some modes of operation, the simulator can act as a turbidostat, such that the density of the simulator algal cultures can be maintained at or below a certain limit, which can be represented, for example, by an optical density (which can be optical density at 730 nm, believed to correlate with cell concentration) to minimize light attenuation through the sample cultures. For example, the desired optical density can be maintained at about 0.6 or less, for example about 0.5 or less, about 0.3 or less, about 0.2 or less, about 0.1, or about 0.1 or less.

Alternatively, in some methods of practicing the invention, the optical density of a simulator culture may be allowed to increase during the sample culturing period. That is, the algae sample may exposed to an illumination profile without dilution of the sample culture during the sample culturing period. For example, an algae sample during a simulation run or sample culturing period can be grown to an optical density of greater than about 0.2, for example, greater than about 0.5, greater than about 1.0, greater than about 1.5, about 2.0, or greater than about 2.0; for example, an algae sample can reach an optical density of between about 0.5 and about 1.0, between about 1.0 and about 1.5, between about 1.5 and about 2.0, or between about 2.0 and about 2.5 during the sample culturing period. In these methods, a simulator culture may be monitored for optical density regularly, such as, for example, daily, during the sample culturing period, and an updated optical density of the simulator culture can be programmed into the simulator system to modify the illumination profile, taking into account the new optical density value. In this way, the simulator sample culture can provide an updated value or values for optical density for the culture as the optical density increases with time, such that the light attenuation model (and illumination profile) can be modified to reflect increases in culture density that occur in a growth environment. Thus, in some examples the algae sample can, during the sample culturing period, be exposed to a series of illumination profiles, as the initial illumination profile is modified to incorporate successive updated optical densities into light attenuation calculations that affect the light intensity values of the illumination profile. The algae sample can thus be exposed to one or more modified illumination profiles, where the modified illumination profiles are based on optical densities of the culture over the sample culturing period.

### Testing Apparatus—Algae Growth Vessel

In order to expose algae to a desired illumination profile, the algae can be placed in a suitable tank or vessel. In addition to allowing for exposure of algae to illumination, the growth vessel can also allow for control of one or more additional variables related to algae growth, such as but not limited to temperature, salinity, pH, carbon dioxide concentration, oxygen concentration, and/or nitrogen concentration.

One set of considerations for the growth vessel can be the size and shape of the vessel. The size and shape of the vessel should be selected so that the vessel can hold a sufficient volume of algae to allow for desired characterization or testing of the algae. Additionally, the vessel can have a shape that reduces or mitigates light intensity attenuation for the algae inside the vessel.

In order to reduce or mitigate light attenuation, the algae-containing water sample within the growth vessel can have a gauge dimension, which can be referred to as a water depth or sample depth, of about a few centimeters or less, such as less than about 10 cm, and preferably less than about 8 cm, for example, less than about 6 cm, less than about 4 cm, less than about 3 cm, between about 2 cm and about 3 cm, about 2 cm, between about 1 cm and about 2 cm, or about 1 centimeter. At a depth of about 1 centimeter, an algae-containing water sample in the vessel can have an optical density of up to about 0.1 or 0.2 while still allowing the algae in the vessel to experience a light intensity comparable to the incident intensity. At a water or sample depth of about 1 centimeter, optical densities up to about 0.5 may also be suitable while still providing a growth environment comparable to a body of water that is being modeled. At a water or sample depth of about 2.5 cm or less, such as about 2 cm or less, optical densities up to about 2.5, for example, up to about 2.0, up to about 1.5, up to about 1.0, or up to about 0.6, may also be suitable. Using methods provided herein, sample cultures having depth dimensions of up to at least about 2 cm and having optical densities greater than 0.6 or greater than about 1.0 can provide a growth environment that results in biomass or biomolecule productivities comparable to a reference body of water that is being modeled. For example, in some instances, algal cultures in the growth vessel having a depth of about 2 cm or less may reach optical densities of greater than 0.2, for example, greater than 0.5, greater than 1.0, between about 1.0 and about 1.5, greater than 1.5, between about 1.5 and about 2.0, or at least about 2.0, during the course of the growth experiment. In some examples, the algal sample may not be diluted during the culturing period.

Note that the “depth” dimension of the growth vessel does not have to be oriented to match the direction of gravitational pull. Instead, the depth or gauge dimension is defined as a direction that is approximately perpendicular to a surface of the water that receives the majority of incident light intensity. For an outdoor pond system, the depth dimension will often coincide with the direction of gravitational pull, but in a closed photobioreactor type system the surface of the water that receives incident light can have any desired orientation relative to gravity. Note that in many cases it can be desirable to use an algae-containing water sample with a sample volume that is less than the volume of the growth vessel. For a growth vessel where the “depth” dimension is roughly aligned with the direction of gravitational pull, the difference between the sample volume and growth vessel volume can result in the depth dimension for the interior of the growth vessel being different from the sample depth dimension for the water sample within the vessel.

In other embodiments, other depths (or gauge dimensions) for the algal culture sample in the growth vessel can be selected, and a corresponding depth can be selected for the interior of the growth vessel. For example, the water or sample depth can be selected so that light attenuation at a desired algae concentration will be acceptable. The water in the growth vessel can have a depth of about 1 cm or less, for example about 2 cm or less, about 5 cm or less, about 8 cm or less, or about 10 cm or less. Additionally or alternatively, the water can have a depth of at least about 0.1 cm, for example at least about 0.2 cm, at least about 0.5 cm, at least about 1 cm, at least about 2 cm, or at least about 4 cm, at least about 6 cm, at least about 8 cm, about 10 cm, or at least about 10 cm.

Another way of selecting a depth for the growth vessel (or depth of the sample in the growth vessel) can be based on the product of the vessel (sample) depth and the expected optical densities for algae solutions that will be studied in the growth vessel. For example, for an algae sample with a depth (gauge dimension) of 0.5 cm and an optical density of 0.3, the product of the sample depth and optical density is 0.15 cm. In various embodiments, the product of the water or sample depth (measured in centimeters) and optical density of the algae sample in the vessel can be about 1.0 cm or less, for example, about 0.75 cm or less or about 0.5 cm or less. In further examples, the product of the water or sample depth in the simulator vessels and the optical density of the algae sample in the vessel can be about 10 cm or less, for example, about 8 cm or less, about 6 cm or less, about 4 cm or less, or about 2 cm or less. In such instances, the product of the water or sample depth (measured in centimeters) and optical density of the algae sample in the vessel can be greater than about 1.0 cm, and may be between about 1.0 cm and 2.0 cm, between about 2.0 cm and about 2.5 cm, between about 3.0 cm and about 3.5 cm, between about 3.0 cm and about 4.0 cm, at least about 4.0 cm, or between about 4.0 cm and about 6.0 cm. The product of the sample depth and optical density can be evaluated based on the initial optical density of an algae sample, or the product of the sample depth and optical density can be used as a maximum value for any time during illumination of an algae sample.

Alternatively, the algal sample depth can be selected such that throughout the sample culturing period the percent transmittance ((I/I₀)×100%) at a PAR wavelength of the algal culture can be at least 35%, for example, at least 40%, at least 45%, or at least about 50%. Alternatively or in addition, the algal sample depth can be selected such that throughout the sample culturing period the percent absorbtion ((I₀₋I/I₀)×100%) at a PAR wavelength of the algal culture can be no higher than about 65%, and preferably no higher than about 60%. For example, the depth dimension can be determined by calculating that, for an algal sample reaching an optical density of between about 0.5 and 1.0, between about 1.0 and about 1.5, or between about 1.5 and about 2.0, the absorbtion can be less than or equal to about 65%, less than or equal to about 60%, or less than or equal to about 50%, and may be less than or equal to about 45%, or less than or equal to about 40%.

The volume of the growth vessel can be selected to hold a desired sample volume of algae-containing water or growth media. One factor in selecting a desired volume of algae-containing water can be the amount of algae that is needed for performing a desired characterization on the algae. For example, an optical density of about 0.1 will correspond to an algae density of about 0.03 g/L to about 0.05 g/L for some types of algae. One way of characterizing the growth rate of algae can be to measure the ash free dry weight of the algae. For this type of measurement to be reproducible, an average sample should contain at least a few milligrams of algae, such as at least about 0.01 g of algae. Thus, for measuring algae growth for an optical density of about 0.1, it is generally beneficial to have an algae sample volume (and therefore a corresponding growth vessel volume) of at least about a liter.

Alternatively, in some methods provided herein, the algae in the test vessel can be grown to a higher density, for example to an optical density (e.g., at ˜730 nm) of 0.5 or greater, for example 1.0 or greater or 1.5 or greater. In these methods, significantly smaller algal test culture volumes can be used, such as, for example, volumes of 500 mL or less, 250 mL or less, 100 mL or less, or 50 mL or less.

In one embodiment, a suitable cross-sectional area for the vessel can be selected based on a desired vessel depth and a desired volume. For convenience, the growth vessel can have a rectangular type cross-section, with the length and width selected to provide a desired volume. For a vessel depth of about 1 cm and a volume of about 1 liter, this corresponds to a square vessel with approximate dimensions of 31.6 cm×31.6 cm×1 cm. This also corresponds to a rectangular vessel with approximate dimensions of 20 cm×50 cm×1 cm. Additionally, where the vessel depth exceeds 2 cm, and the culture density times the culture depth exceeds 1 cm, the height and width dimensions of the vessel can be considerably smaller, as the culture volume needed for productivity assessment is less. For example, a rectangular vessel having a volume of approximately 150 mL can have approximate dimensions of 8 cm×12.5 cm×2 cm, where 1.9 cm is the oriented as the depth dimension. Alternatively, any other convenient cross-section can be selected, such as a cross-section that facilitates even distribution of light intensity across the surface of the vessel based on the geometry of the illumination source for the growth vessel. Thus, circular, trapezoidal, or other regular or irregular shapes can be selected for the growth vessel.

In another embodiment, the growth vessel can comprise two chambers, designated the “growth chamber” and the “selection chamber.” In some embodiments, the optical depth of the growth chamber should be at least about ten times greater than the optical depth of the selection chamber. In some embodiments, the growth chamber and the selection chamber are hydraulically connected, optionally with a valve controlling flow through the hydraulic connection. In certain embodiments, the hydraulic connection comprises a flow cytometer to measure various properties of cells as they move between the growth chamber and the selection chamber. Although the cross section of each chamber can come in any shape, in some embodiments, the shape will be rectangular for convenience of storage and operation. In some embodiments, one or both chambers comprise means for controlling various culture parameters, such as temperature, salinity, carbon dioxide concentration, oxygen concentration, nitrogen concentration, and/or pH. In certain embodiments one or both chambers, but especially the selection chamber, will comprise a port for sampling off-gas from the cultures.

For ease of use, it may be desirable to have a growth vessel with a volume that is larger than the desired sample volume of algae containing water. For example, a suitable rectangular vessel can have approximate dimensions of 25 cm×50 cm×1.3 cm, which correspond to an internal volume for the growth vessel of ˜1.625 liters. During operation, about 1.4 liters of algae-containing water or growth media can be used. In this embodiment, the depth or gauge dimension of the vessel corresponds to the 1.3 cm. The vessel can be oriented so that either the 25 cm or the 50 cm dimension is oriented approximately in the direction of gravitational pull. This can result in the depth dimension being oriented roughly perpendicular to the direction of gravitational pull. The light source for the growth vessel can be located and oriented so that the incident light passes through a surface of the growth vessel having the 25 cm×50 cm cross-sectional area. In this type of configuration, placing about 1.4 liters in a ˜1.625 liter vessel will result in an unoccupied volume in the vessel. This unoccupied volume does not change the definition for what is considered the depth dimension. As defined above, the depth dimension corresponds to the dimension roughly perpendicular to the surface that receives the majority of incident light intensity. Those of skill in the art will clearly recognize that the depth or gauge dimension in this embodiment corresponds to the 1.3 cm dimension.

In additional examples, for example, for use in methods where algae are allowed to grow to densities of 0.5 or greater, smaller volume vessels may be used, for example, where the depth dimension may be 2.0 cm or less, and the vessels may hold up to 500 mL, for example, up to 400 mL, up to 300 mL, up to 200 mL, up to 100 mL, up to 50 mL, up to 40 mL, or less than 40 mL, of algal culture. For example, standard disposable essentially-rectangular tissue culture flasks may be used, and can have dimensions of approximately 12.6 cm×7.8 cm×1.9 cm and an internal volume of approximately 150 mL. The flasks may contain, for example, approximately 100 mL of algal culture during the culturing period.

To allow in light, the vessel should be constructed of a material that is transparent or substantially transparent to the incident light used for illuminating algae in the vessel. Suitable materials for the container can include various types of clear glass or plastic. Clear polycarbonate plastic is one useful structural material, as polycarbonate facilitates sterilization of the growth vessel prior to the beginning of a test. It is noted that ultraviolet light is typically not involved in photosynthesis reactions, so UV attenuation due to the structural materials can be acceptable. As an alternative, any attenuation of light by the structural material for the growth vessel can be accounted for by using a correspondingly stronger illumination source, so that the light intensity incident on the surface of the algae-containing water can approximately match the desired model intensity from an illumination profile.

The growth vessel can include other features to allow for control of the reaction conditions in the vessel. For example, the temperature in the growth vessel can typically also be controlled, as algae growth rates are often strongly influenced by temperature. If a relatively stable temperature is desired for the growth vessel, the growth vessel can be placed in a sand bath, with the back side of the vessel in contact with the sand. Another method for controlling the temperature in the growth vessel can be to use one or more thermoelectric heaters. The thermoelectric heaters can be attached to and/or incorporated into a sidewall of the growth vessel. Optionally, the thermoelectric heaters can be located on a sidewall so that the heaters are not in the path for light incident on the algae. When the target temperature for the vessel is higher than the current temperature, the thermoelectric heaters can be used to increase the temperature. When the target temperature is lower than the current temperature, the heaters can be turned off to allow the vessel to cool in a passive manner.

Heating and cooling of the growth vessel can be used to represent the heating and cooling a pond would experience due to exposure to external conditions. A raceway pond can have a depth of only a meter or less, and typically only a few tens of centimeters or less, and therefore a raceway pond may have a relatively constant temperature at any given time. However, the temperature of the pond may vary during the course of a trajectory. For example, during daylight hours a pond can increase in temperature based on incident radiation. Any sunlight that is incident on the pond surface can be assumed to be reflected at the surface, used for algae photosynthesis, or absorbed by the pond and converted to heat. Optionally, the contribution of sunlight to algae photosynthesis can be ignored to simplify the calculation. The pond can also lose heat to the environment via convection, radiative transfer, and other mechanisms. This heat exchange can be modeled to provide a temperature profile for the pond that is correlated with the illumination profile. In other words, each time in the trajectory can have both an illumination value and a temperature value. Note that the illumination and temperature values associated with a time in a trajectory may be illumination and temperature values that span a period of time. For example, a temperature value and/or illumination value may be specified for each 1 second period, each 5 second period, each 10 second period, each 1 minute period, and/or any other convenient period of time. When algae in a growth vessel is exposed to illumination based on an illumination profile, the water in the growth vessel can then be heated or cooled accordingly based on the correlated temperature profile.

Another factor that can be controlled in the growth vessel is the pH of the water. In many embodiments, CO₂ can be the primary acidic component in the algae-containing water, and therefore the pH can be controlled by controlling the CO₂ content. CO₂ can be introduced into the growth vessel via an inlet that allows for bubbling CO₂ into the vessel. Alternatively, an aeration port can be used to introduce CO₂. A flow meter or another convenient device can be used to control the input flow rate of CO₂ into the growth vessel. During exposure of algae to an illumination profile, it may be desirable to hold the CO₂ concentration at a relatively constant value. Alternatively, if a pond is being modeled that has one or a few discreet CO₂ input sources, the CO₂ concentration experienced by the algae may vary as the algae traverses the pond. Thus, it can be desirable to vary the CO₂ concentration during the course of an illumination profile.

Still another factor that can be controlled in the growth vessel is the oxygen content. Algae can produce molecular oxygen as a by-product of photosynthesis. An inlet can be included in the growth vessel to allow for addition of oxygen in order to match a model oxygen content. An aeration port can also be included to allow for removal of oxygen in order to match a model oxygen content.

Because nitrogen content can have an important influence on biomass production, it can be important in addition to control nitrogen content of the medium. Although those skilled in the art will be familiar with the many well-known methods for controlling nitrogen content of a culture, perhaps the simplest and most efficient is simply to add nitrogen salts when more high-nitrogen conditions are desired, and to dilute nitrogen rich cultures with nitrogen-free, defined medium when lower nitrogen conditions are desired.

In addition to inputs to the growth vessel, the growth vessel can also include features to allow for movement of water or growth media within the growth vessel. In examples where the sample volume is small, for example, about 1 liter or less, bubbling of air or CO₂ through the culture, for example, by insertion of a tube into the culture, can provide adequate culture mixing. An alternative or additional option can be to include a mechanical agitator in the growth vessel, to increase mixing within the vessel. One or more internal dividers can be included in the growth vessel, such as dividers that can be used to set up a flow path within the vessel. A mechanical paddle wheel or another mechanism can then be used to create a flow within the growth vessel. In further embodiments, a sparging mechanism can be used to provide movement or agitation of the water/growth medium in the growth vessel. As an example, a sparging mechanism could be used for introduction of CO₂ into the growth vessel.

In other embodiments, different types of growth vessel may be suitable. For example, another type of screening test can be a test of lipid production. It is noted that the algae may undergo little or no growth during a lipid production test. During a lipid production test, a simplified vessel with a smaller volume may be suitable. The vessel may not need to include inlets or aeration for providing or removing gases during a test run. Instead, the vessel can allow for introduction of an algae sample with a desired initial pH and O₂ content. The vessel can then be closed during performance of the test. The temperature can be controlled by any convenient method, such as by using a sand bath or thermoelectric heating. The vessel can then be exposed to an illumination profile for lipid production.

### Testing Apparatus—Illumination Source

After generating an illumination profile, an algae sample in the growth vessel can be exposed to the illumination profile by controlling the output of an illumination source. In various embodiments, the illumination source can be capable of delivering light intensity to the surface of the water sample in the growth vessel. The intensity can range from no illumination to an intensity that is at least about the maximum intensity in the illumination profile. The illumination source can be controlled to vary the delivered intensity in accordance with the illumination profile.

One consideration in selecting an illumination source can be the maximum light intensity that is required within an illumination profile. For example, an illumination profile based on sunlight incident on a pond can have a maximum incident intensity corresponding to a maximum incident sunlight intensity. Depending on the modeled location for the pond, this can be up to about 2400 μE/m²/sec PAR. Certain synthetic trajectories can require sustained intensities of light that are more intense than sunlight, such as light intensities of about 4800 μE/m²/sec PAR, about 7200 μE/m²/sec PAR, about 9600 μE/m²/sec PAR, or even about 12000 μE/m²/sec PAR. Preferably, the illumination source can deliver light at 7200 μE/m²/sec to the surface of the sample in the growth vessel. In some embodiments, an illumination source capable of delivering a greater intensity can be used, and then scaled back to match the intensity specified in the illumination profile. In certain embodiments, the illumination source is designed to provide illumination that is substantially or totally actinic light. Most known wild-type algae have a saturation intensity of about 1000 μE/m²/sec PAR or less. Thus, for some synthetic trajectories, a light source having an intensity of at least 1000 μE/m²/sec PAR is sufficient, such as about 1100 μE/m²/sec PAR. For example, consider a rectangular vessel having a cross-sectional area of 20 cm×50 cm. This corresponds to 0.1 m² of surface area for the algae-containing water in the growth vessel. In order to deliver about 1000 μE/m²/sec PAR to this surface area, an illumination source can be used that can deliver at least about 100 μE/sec PAR. In various embodiments, the illumination source can deliver to the surface of the vessel at least about 40 μE/sec PAR, for example, at least about 80 μE/sec PAR, at least about 100 μE/sec PAR, or at least about 150 μE/sec PAR. Additionally or alternately, the illumination source can deliver to the surface of the vessel about 240 μE/sec PAR or less, for example, about 200 μE/sec PAR or less, about 150 μE/sec PAR or less, or about 120 μE/sec PAR or less.

One option for the illumination source can be to use a plurality of light emitting diodes (LEDs). LEDs typically generate non-collimated light, so the LEDs can be used in conjunction with lenses in order to direct the light toward the surface of the water. One feature of some LED light sources is that an LED can have a defined relationship between input power and output illumination over a range of LED outputs. This can allow an LED to deliver a percentage of the input power as output illumination. In the example described above, if an LED has 40% efficiency, a sufficient number of LEDs can be used so that the input power to the LEDs can be about 35-40 W, so that the output power can be at least about 14 W. This can correspond to a light intensity for the LEDs of about 125-150 μE/s PAR (depending on the nature of the LED). The input power to the LED can be regulated by controlling the current delivered to the LED, such as by using a commercially available constant current controller. Because LEDs respond quickly to changes in current, it can often be desirable to use a constant current controller to vary LED output in accordance with an illumination profile. Exemplary arrangements of lights, lenses, and vessels can be found in U.S. 2013/0143255, the entire contents of which are herein incorporated by reference.

In an embodiment, an array of LEDs is used that are arranged roughly in the same geometry as the cross-section of the growth vessel, such as a rectangular array for a rectangular growth vessel cross-section. A square shaped pattern lens can be used in front of each LED to approximately direct the light onto the surface of the water in the growth vessel. The LEDs in the array can be selected to have roughly the same light output, and the LEDs can be spaced in a regular pattern to generate a roughly uniform illumination of the water surface in the growth vessel. In other embodiments, other choices for number, type, and spacing of LEDs can be used to generate a desired illumination pattern on the surface of water in a growth vessel.

The algal growth simulation system in many examples will be used to replication growth conditions of a pond or a photobioreactor exposed to sunlight. In replicating these reference embodiments, the light source can be arranged such that the growth vessel is illuminated from a single direction. Thus a typical design that employs a light source such as an array of LEDs can have the LED array positioned on a single side of the growth vessels, i.e., facing the cross-sectional area of the growth vessel referred to above, where light is directed through the depth of the culture.

In some embodiments, the color of the LEDs can be selected to approximate the color spectrum of the light incident on the body of water being modeled. If the modeled light source is sunlight, a white LED can be selected. The output of a white LED will likely have a different mixture of wavelengths than sunlight, but it is not believed to be necessary to exactly match the light source being modeled. In alternative embodiments, an LED with a more limited color spectrum can be used, such as a red or a blue LED.

Because of the intensity requirements, it can be beneficial to use an LED that can convert input energy to light with relatively high efficiency. This is not essential, but it can help with assembling a sufficient number of LEDs to provide the desired incident intensity in a reasonable amount of area. Examples of suitable LEDs are available from Phillips Lumileds Lighting Co. and Cree, Inc.

In order to deliver a desired amount of illumination to an algae sample, the illumination delivered at various power levels by a light source array (such as an LED array) can be measured. This can allow for calibration of the light source array in advance. Alternatively, if an existing relationship is known between input power and output illumination, the light source array can be used based on the expected relationship.

### Operating Conditions—Algae Growth

Algae for use in the methods and systems of the present invention can be grown in any suitable culture medium, including media well known to those of skill in the art. Solid and liquid growth media are generally available from a wide variety of sources, as are instructions for the preparation of particular media suitable for a wide variety of strains of microorganisms. For example, various fresh water and salt water media can include those described in Barsanti, L. and Gualtieri, P. (2005) Algae: Anatomy, Biochemistry, and Biotechnology, CRC Press, Taylor & Francis Group, Boca Raton, Fla., USA, which is incorporated herein by reference for media and methods for culturing algae, or in the examples of PCT Publication No. WO 2008/151149, which is herein incorporated by reference. Algal media recipes can also be found at the websites of various algal culture collections, including, as nonlimiting examples, the UTEX Culture Collection of Algae (sbs.utexas.edu/utexmedia.aspx); Culture Collection of Algae and Protozoa (ccap.ac.uk/media/pdfrecipes.htm); and Katedra Botaniky (/botany.natur.cuni.cz/algo/caup-media.html). One useful culture medium is BG-11, the ingredients of which are given in Table 1. Another useful culture medium is Guillard's f/2 culture medium, supplemented with nitrogen, phosphorus, and iron.

In some methods, a desired concentration of algae can be introduced into the growth vessel. The concentration of algae can be selected based on a desired optical density for the algae and water in the growth vessel. The desired optical density can be at least about 0.01, for example at least about 0.05, at least about 0.1, or at least about 0.2. The desired optical density can additionally or alternatively be about 0.6 or less, for example about 0.5 or less, about 0.3 or less, about 0.2 or less, or about 0.1 or less. Although an optical density of about 0.5 can lead to some light attenuation even in a vessel with a depth of 1 cm, it is believed that results generated at this type of optical density can still be representative of a reference system.

TABLE 1
BG-11 Medium (ATTC)

| NaNO₃ | 1.5 | g |
|---|---|---|
| K₂HPO₄ | 0.04 | g |
| MgSO₄ * 7H₂O | 0.075 | g |
| CaCl₂ * 2H₂O | 0.036 | g |
| Citric acid | 6.0 | mg |
| Ferric ammonium citrate | 6.0 | mg |
| EDTA | 1.0 | mg |
| Na₂CO₃ | 0.02 | g |
| Trace Metal Mix A5* | 1.0 | ml |
| Agar (if needed) | (up to) 10.0 g | |
| Distilled water | 1.0 | L |

*Trace Metal Mix A5 Composition

| | |
|---|---|
| H₃BO₃ | 2.86 g |
| MnCl₂ * 4H₂O | 1.81 g |
| ZrSO₄ * 7H₂O | 0.22 g |
| Na₂MoO₄ * 2H₂O | 0.39 g |
| CuSO₄ * 5H₂O | 0.080 g |
| Co(NO₃)₂ * 6H₂O | 49.4 mg |
| Distilled water | to 1.0 L |

In some embodiments, the depth of the growth vessel can be selected based on a desired optical density for simulation. For example, if it is desired to measure algae growth at an optical density of about 0.1, a vessel having dimensions of about 20 cm×50 cm×1 cm may be suitable. If it is desired to measure algae growth at an optical density of 0.5, a similar vessel could be used, or a vessel having dimensions of 40 cm×50 cm×0.5 cm may be suitable. Similarly, if a more dilute solution of algae is desirable, a vessel with a greater depth than 1 cm may be appropriate.

Algae in the vessel can then be exposed to an illumination profile for a period of time, referred to herein as the sample culturing period, such as an hour, multiple hours, a day, multiple days, or even months. As the algae grow, the concentration of algae in the growth vessel can increase, which can lead to an increase in optical density. For testing of growth at a given optical density, the sample in the vessel can be diluted on a periodic basis to return the growth vessel to the original optical density. For example, on a daily basis water in the vessel can be sampled and the optical density can be measured, such as in a spectrophotometer. Based on the measured optical density, the algae in the vessel can be diluted to return the optical density to the desired optical density. The water can be sampled again to verify that the desired optical density has been achieved. This technique can allow for simulation of a long period of growth while maintaining an optical density that does not result in excessive attenuation.

In alternative methods, the optical density of a simulator culture may be allowed to increase during the sample culturing period. In these methods, the algae sample may be exposed to an illumination profile without regular dilution of the sample culture during the sample culturing period. This can allow for smaller volume sample cultures, as the biomass of the algae sample increases with the optical density. For example, the optical density of the algal sample can be allowed to reach a value of at least about 0.3, at least about 0.5, at least about 1.0, or greater, such as an optical density of between about 0.5 and about 1.0, between about 1.0 and about 1.5, between about 1.5 and about 2.0, at least about 2.0, between about 2.0 and about 2.5, or greater than about 2.5, during the sample culturing period. For example, the maximal optical density during the culturing period can be about 3.0 or less, for example about 2.5 or less, about 2.0 or less, about 1.5 or less, about 0.8 or less, about 0.6 or less, about 0.5 or less, or about 0.3 or less. Although an optical density of about 0.5 or greater can lead to some light attenuation even in a vessel with a depth of 1 cm, it is demonstrated herein that results generated at optical densities of greater than 0.5 and sample depths of greater than 1.0 cm can still be representative of a modeled system such as a mixed pond or bioreactor.

For example, the simulation can be run where the cultures are allowed to reach an OD of at least about 0.3 or at least about 0.5, such as between about 0.5 and about 1.0, between about 1.0 and about 1.5, between about 1.5 and about 2.0, between about 2.0 and about 2.5, or between about 2.5 and about 3.0, and the growth vessel can be between about 0.5 cm and about 1.0 cm in width, for example between about 1 cm and about 1.5 cm in width, between about 1.5 cm and about 2 cm in width, or between about 2.0 cm and about 2.5 cm in width. The other dimensions of the vessel may be reduced accordingly to provide a convenient volume of culture having a biomass of at least about 10 mg at the end of the simulator run.

In such examples, the sample cultures can have a volume of, for example, less than about one liter, less than about 500 mL, less than about 200 mL, less than or equal to about 100 mL, or less than or equal to about 50 mL. In these methods, a simulator culture can preferably be monitored regularly, such as daily, for optical density, and the optical density of the simulator culture can be programmed into the simulator system to modify the illumination profile, taking into account the updated (i.e., most recent) sample optical density value which is used in calculating light attenuation. In this way, the simulator culture can provide one or more updated values for optical density that can be used in modeling the light attenuation of an algal reference culture over time, which can be used to provide a modified illumination profile, such as a first modified illumination profile, and the algae sample can then be exposed to the (first) modified illumination profile as the culturing period continues. Further, until a second updated optical density can be measured and a second modified illumination profile can be calculated and applied to the sample culture. The modified light attenuation profile(s) can be based on optical density increases in the sample that may model increases in culture density that occur in a growth environment, such as the modeled reference growth environment.

For example, in these methods, the algae can be allowed to grow without dilution for two, three, four, five, six, seven or more days, or for the same amount of time as they would be expected to grow in a reference volume (e.g., a mixed pond), where, in some embodiments, the cultures are not diluted during this culture period. Optical density measurements of the sample cultures can be taken daily or at any convenient time interval, and these updated optical density values can be programmed into the simulator system to modify the light intensity profile as the simulation sample culturing period progresses, to more accurately model increased light attenuation based on increased density of an algal culture over time. Thus, in these methods, the algae sample can be exposed to an initial illumination profile that is subsequently modified based on measurements of optical density of the sample cultures, whereupon the algae sample is exposed to a modified illumination profile that takes into account the updated optical density. Illumination profiles may be modified at regular intervals, such as, for example, daily, based on optical density measurements taken at regular intervals, e.g., daily. For example, an algae sample can be exposed to a first illumination profile at a first stage of the sample culturing period, and can subsequently be exposed to a second modified illumination profile during a second stage of the sample culturing period, where the second illumination profile is calculated using a second updated optical density measurement taken during the sample culturing period. The algae sample can be exposed to one, two, three, four, five, six, seven, eight, nine, ten, or more modified illumination profiles during the sample culturing period, where successive modified illumination profiles can be based on successive updated algae sample optical densities measured during the sample culturing period.

In certain embodiments, the maximum optical density of the algal sample during the scale-down sample culturing period allows for a culture absorbtion ((I₀−I_{L}/I₀)×100%) at a PAR wavelength of at least about 30%, for example, at least about 35%, at least about 40%, or at least about 45%. For instance, the culture absorbtion can reach a value of between about 35% and about 40%, between about 40% and about 45%, between about 45% and about 50%, between about 50% and about 55%, or between about 55% and about 60%. The transmittance (I_{L}/I₀) of the algal sample can preferably be maintained at least 35%, and more preferably at least 40%, at least 45%, or at least 50%, during the culture period. Using Beer's Law as provided above, where absorbance=αL=−log₁₀(I_{L}/I₀)=εLc, it can be seen that the transmittance can be maintained at a level at or above 35% by adjusting the depth of the flask (L) and/or the optical density of the culture.

In instances where an algal sample cultured in a algal growth simulator system reaches a density of greater than about 0.3, such as greater than about 0.4, greater than about 0.5, or at least about 0.6, the culture vessel can have reduced dimensions with respect to the examples above. For example, a culture vessel can be 10 cm or less×25 cm or less×1 cm or 8 cm×15 cm×2 cm, etc. The ability of a culture vessel to simulate growth of algae in the reference volume can be determined empirically, and can be aided by determining or approximating the extinction coefficient profile of the algal strain or a taxonomically related strain.

After a desired amount of exposure of algae to an illumination profile (or a series of illumination profiles, where an initial illumination profile is followed by one or more modified illumination profiles) one or more tests can be performed on the algae to identify samples with traits correlating with or indicative of improved photosynthetic efficiency. For example, in certain embodiments it may be useful to measure pigment concentration (such as chlorophyll concentration), oxygen evolution, carbon fixation, and/or tolerance of cellular membranes (such as the membranes of the chloroplast) to various levels of electrical potential. In certain embodiments it can be useful to calculate the specific growth rate of one or more strains in the culture over a period of time.

In certain embodiments, it may be useful to measure the ash free dry weight of an algae sample. Measuring the ash free dry weight can provide a value for the growth rate of an algae sample relative to a starting algae concentration. To measure the ash free dry weight, at least a portion of the algae sample in the vessel can be withdrawn. The withdrawn sample can be filtered to separate solid matter from the surrounding water. The sample can then be further dried to remove additional water. The additional drying can include heating of the sample, but combustion of any portion of the sample should be avoided. The dried sample can then be weighed. After determining a pre-combustion weight, the sample can be combusted. The ash remaining after combustion can be dried and then weighed. The difference between the initial weight and the amount of ash (the ash free dry weight) provides a numerical value for the amount of biomaterial in the original sample.

Another type of measurement can be a measurement of the total organic carbon in an algae sample. Many variations for total organic carbon analysis are available. Methods for total organic carbon analysis typically involve an initial acidification of a sample to drive dissolved CO₂ out of the sample. The sample can then be combusted or oxidized by various methods, and the CO₂ evolved from combustion/oxidation can be measured as an indication of carbon content. The evolved carbon can be measured by measuring a conductivity of the sample before and after evolution of CO₂, or by non-dispersive infrared analysis. For example, total organic carbon for an algal sample can be analyzed using a Shimadzu TOC-VCSH Analyzer, which efficiently oxidizes organic compounds.

Still another type of characterization can be lipid productivity. For example, the total amount of lipids present in an algae sample can be measured by fatty acid methyl ester (FAME) analysis, in lipids from algae are determined as fatty acid methyl esters by gas chromatography. Lipid productivity can be useful for measuring the capability of an algae sample for generating the lipid products which can eventually be converted into a diesel fuel or other valuable product. For methylation of free fatty acids and transmethylation of lipids the AOCS method Ce 1j-07 is used with some modifications, followed by alkali hydrolysis and methylation.

To determine the fatty acid ester content by FAME analysis, an algal culture sample (˜2 mL) can be lyophilized to dryness followed by alkali hydrolyses with ˜700 uL of ˜0.5M KOH in methanol/tetrahydrofuran (˜2.5:1) mix. Glass beads can be added to the tubes, which can then be vortexed and then heated at ˜80° C. for ˜5 mins. The tubes can be allowed to cool ˜5 mins at room temperature (˜20-25° C.), before methylation with ˜500 μL of ˜10% BF₃ at ˜80° C. for ˜30 mins. Vials can then be allowed to cool ˜5 mins before extraction with ˜2 mL of heptane and ˜500 uL of ˜5M NaCl. After vortexing, samples can be centrifuged for ˜1 min at ˜2000 rpm to separate phases. About 0.9 μl of the hexane extract can be injected into an Agilent 7890A gas chromatography system at a flow rate of ˜0.5 mL/min hydrogen at ˜100° C. for about 1 min, followed by a relatively fast temperature gradient to ˜230° C. for ˜1.7 mins. A DB-FFAP capillary column (J&W Scientific) can be used, ˜10 m long with ˜0.10 mm diameter and ˜0.10 m film thickness. The inlet can be held at ˜250° C., and the FID detector at ˜260° C.

Peaks can be identified based on external standards. Absolute areas for both analytes and the internal standards can be obtained and the amount of FAME calculated for each sample. The efficiency of derivatization of triacylglycerides can be determined by computing the ratio between FAME originating from a triacylglyceride internal standard (e.g., C13:0) and FAME originating from a FAME internal standard (e.g., C23:0). The efficiency of derivatization of fatty acids can be determined by computing the ratio between FAME originating from an internal standard free fatty acid (e.g., C11:0) and FAME originating from an internal standard FAME (e.g., C23:0) (The ratios should be close to 1.).

To determine the total organic carbon (TOC) content of algal cells, samples of cell cultures can be centrifuged to remove media and resuspended in water. Cell samples (three per measurement) can be injected into a Shimadzu TOC-Vcsj Analyzer for determination of Total Carbon, Total Inorganic Carbon, and, optionally, Total Nitrogen. The combustion furnace can be set to ˜720° C., and TOC can be determined by subtracting TIC from TC. The calibration range can be from ˜2 ppm to ˜200 ppm. The correlation coefficient requirement is preferably r²>0.999.

In addition, scale-down cultures can be tested for photosynthetic properties, including, for example, Fᵥ/Fₘ, oxygen evolution, and non-photochemical quenching. The scale-down cultures can be used to assay for or perform chemical analysis to detect metabolites, pigments, particular lipids, cofactors, or enzymes. The scale-down cultures can also be tested for expression of particular genes or production of proteins, for example using PCR, nucleic acid hybridization, antibody detection, or other techniques.

The system can include more than one growth vessel, and can test replicate cultures of a strain, optionally can test more than one growth condition for a strain, and can test more than one strain of algae during a single run. In some examples, algal mutants and parent wild-type strains can be tested together, where the algal mutants and the parent strain have similar molar absorptivities. In other examples, different sub-species of the same species can be tested together. The productivities of strains can be assessed and compared with one another based on productivities or growth rates, for example, or biochemical or molecular genetic assays or analysis can be performed on the cells of the simulator cultures. The results of growth, productivity, biochemical, or molecular genetic (e.g., gene expression) analysis can be used to screen and/or compare algal strains and mutants, including genetically engineered strains, in a simulated environment prior to or in lieu of, testing in large volume growth systems. In certain embodiments the various strains in co-culture can grow under selective conditions that will favor the growth of one strain over another.

### Alternative Configurations—High Throughput Testing

The above systems and methods provide examples for simulating algae growth and/or lipid production in a larger body of water (such as a photobioreactor or pond) using a small scale system. In other embodiments, algae growth and/or lipid production can be simulated in multiple vessels in parallel at the same time. If sufficient space is available, multiple vessels of any convenient size, for example, a size of from 100 microliters to 1 liter, or any other convenient size, can be exposed to illumination profiles in parallel. For example, multiwell plates can be used, or can be adapted for use, in which in various nonlimiting embodiments the wells of a 96-well, 48-well, 24-well, 12-well, or 6-well multiwell plate can contain, for example, 100 microliters, 200 microliters, 1 milliliter, 2 milliliters, 3 milliliters, 4 milliliters, or 5 milliliters of water or culture media. Alternatively, multiple smaller vessels can in some embodiments be exposed to illumination to test for features that can be measured with a smaller sample size, such as lipid productivity.

One way to enable high throughput testing can be to have multiple banks of LEDs that can be controlled separately as an illumination source. Using lenses, the light from each bank of LEDs can be approximately focused to impinge on one or more selected growth vessels. For example, consider an illumination device with four banks of LEDs. The banks can be arranged in rows. Each row can be used to provide a different illumination profile. One or more growth vessels can be positioned to receive illumination from each of the banks. One option could be to have a plurality of growth vessels arranged in columns, with the same type of algae in each vessel in a column. In this manner, a matrix of algae growth and/or lipid production experiments can be run at the same time, as each vessel will represent a unique combination of an algae (arranged by column) and illumination profile (arranged by row). Of course, other ways of arranging light banks and growth vessels will be apparent to those of skill in the art.

### Types of Algae

An algal strain can include any isolate of an algal species or subspecies, and includes mutants and genetically engineered strains. Algae considered herein can include, but are not limited to, unicellular and multicellular algae. Examples of such algae can include a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof. In one embodiment, algae can be of the classes Chlorophyceae and/or Haptophyta, Bacillariophyceae, Eustigmatophyceae, Trebouxiophyceae, or Prasinophyceae. Specific species can include, but are not limited to, Neochloris oleoabundans, Scenedesmus dimorphus, Euglena gracilis, Phaeodactylum tricornutum, Pleurochrvsis carterae, Prymnesium parvum, Tetraselmis chui, Nannochloropsis gaditana, Dunaliella salina, Dunaliella tertiolecta, Chlorella vulgaris, Chlorella variabilis, and Chlamydomonas reinhardii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryplomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrsis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactilum, Phagus, Picochlorum, Platymonas, Pleurochiysis, Pleurococcus, Prototheca, Pseudochlorella, Pseudoneochloris, Pyramimonas, Pyrobotrys, Scenedesmus, Schizochlamvdella, Skeletonema, Spyvrogyra, Stichococcus, Tetrachorella, Tetraselmis, Thalassiosira, Viridiella, or Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cvanobacterium, Cyanobium, Cyanocistis, Cyanospira, Cvanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Sligonema, Symploca, Synechococcus, Synechocytis, Tolypothrix, Trichodesmium, Tychonema, and Xenococcus species.

Algae oils or lipids are typically contained in algae in the form of membrane components, storage products, and metabolites. Certain algal strains, particularly microalgae such as diatoms, certain chlorophyte species, and cyanobacteria, contain proportionally high levels of lipids. Algal sources for the algae oils can contain varying amounts, e.g., from 2 wt % to 40 wt % of lipids, based on total weight of the biomass itself. The scale-down systems and methods described herein can be used to evaluate production of lipids by algae strains.

### Methods for Determining Effects of Parameters on Photosynthetic Efficiency

Also provided herein are methods for determining an effect of a physiological or environmental parameter on algal photosynthetic efficiency. As noted above, the “efficiency” of photosynthesis refers to the percentage of actinic light energy that a given organism encounters and is able to convert into biomass. Therefore, two convenient proxies by which photosynthetic efficiency can be measured are specific growth rate and simulated density of the culture. Specific growth rate and simulated density can be measured by many means, including the means and equations disclosed hereinabove.

In certain embodiments, the importance of a particular variable in a model of photosynthesis can be evaluated by setting that variable at an extreme or exaggerated degree in a growth simulation. For example, methods disclosed herein can be used to determine whether the history of a cell impacts the cells subsequent, instantaneous photosynthetic rate. Similarly, methods disclosed herein can be used to determine the effect that certain dynamic responses to light exposure, such as non-photochemical quenching, have on other dynamic responses, such as antenna growth or recession, and to estimate more accurately the rate constants with which such dynamic responses activate, inactivate, and cross-talk with each other. Non-limiting examples of physiological or environmental parameters whose effects on photosynthetic efficiency can be tested include temperature, pH, carbon concentration, oxygen concentration, and/or nitrogen concentration.

A synthetic trajectory or a plurality of synthetic trajectories that might occur if the variable were set to an extreme or exaggerated degree can be contrived, and illumination profiles formed corresponding to these synthetic trajectories. Cells can then be exposed to the illumination profile or profiles. The trajectories used for purposes of these methods can be a synthetic trajectory, such as an unreal trajectory. In certain embodiments, the unreal trajectory can be a discontinuous trajectory, as defined above. For example, the trajectory can require that a cell move instantaneously between two locations without passing through intermediate locations, as if a cell were 2 cm from the pond surface one second, and 6 cm from the surface the next second, without the cell ever passing through regions 3, 4, or 5 cm from the surface. Additionally or alternatively, the trajectory can require that the cell experience high light intensity (such as at least 150, at least 200, at least 300, at least 500, at least 900, at least 1000, at least 1500 or at least 2000 μE/m²/sec and in some cases up to 3000 μE/m²/sec), followed immediately by darkness, with no intermediate illumination transition. Additionally or alternatively, the illumination profile can be mostly or entirely composed of low light intensities, such as intensities not more than 200 μE/m²/sec, for example not more than 150, not more than 100, not more than 75, not more that 50, not more than 25, or even 0 μE/m²/sec. It will be understood by one of skill in the art that the terms “high” and “low” are implicitly used in reference to a particular organism, and that a light condition that is “low” for one organism can be “high” for another. In some embodiments, the movement between light and dark can be periodic; in other embodiments the movement between light and dark can be quasiperiodic; in yet other embodiments the movement can be irregular, with no pattern in the rate at which the cell transitions from light to dark; in still other embodiments, the movement between light and dark can proceed through discrete, step-wise escalations and de-escalations of illumination intensities. Strains of algal cells, individually or in co-culture, can then be measured or tested to determine the effect of the extreme or exaggerated variable on the photosynthetic efficiency of each strain of algal cells. Such tests can be by any method known in the art and/or discussed herein. In some embodiments, the tested parameter will be assayed for its effect on specific growth rate, simulated culture density, or simulated biomass density.

### OTHER EMBODIMENTS

Additionally or alternately to any of the described methods and systems, the present invention can include one or more of the following embodiments:

### Embodiment 1

A method for selecting a strain of algal cells for biomass accumulation, preferably enhanced biomass accumulation relative to a prior art strain of algal cells, the method comprising: (a) co-culturing at least two strains of algal cells; (b) calculating a particle trajectory for a particle in a reference volume, the particle trajectory comprising at least a plurality of position values in the reference volume, the plurality of position values having continuous associated times, the position values including at least a depth value relative to a light-incident surface of the reference volume; (c) forming an illumination profile based on the particle trajectory by obtaining an illumination intensity corresponding to the plurality of position values and associated continuous times, wherein forming an illumination profile comprises: selecting a depth value and an associated time; selecting an intensity of illumination that is incident on a surface of the reference volume at the associated time; selecting an optical density at the associated time; and calculating an illumination for the selected depth value based on an attenuation of the selected incident illumination intensity and the selected optical density; (d) exposing the co-cultured strains of algal cells in a vessel to light intensity corresponding to the illumination profile, the algae sample having a sample depth, a sample volume, and an optical density, a product of the optical density and the sample depth being less than about 4.0 cm, the sample volume being less than the reference volume; and characterizing at least one algae property for algae in the reference volume based on at least one measured algae property of the algae sample.

### Embodiment 2

A method for prediction of algal behavior in a reference environment based on growth of algae in a controlled environment, comprising: (i) contriving a synthetic trajectory for a particle in a reference volume, the particle trajectory comprising at least a plurality of position values in the reference volume, the plurality of position values having continuous associated times, the position values including at least a depth value relative to a surface of the reference volume; (ii) determining an initial illumination profile based on the particle trajectory by obtaining an illumination intensity corresponding to the plurality of position values and associated times, wherein obtaining an illumination intensity corresponding to the plurality of position values and associated times comprises: selecting a depth value and an associated time; selecting an intensity of illumination that is incident on a surface of the reference volume at the associated time; selecting an optical density at the associated time; and calculating an illumination for the selected depth value based on an attenuation of the selected incident illumination intensity and the selected optical density; (iii) exposing an algae sample in a vessel to light intensity corresponding to the initial illumination profile, the algae sample having a sample depth, a sample volume less than that of the reference volume, and an optical density, wherein the optical density varies over the sample culturing period; (iv) measuring the optical density of the algae culture during the sample culturing period to obtain an updated sample optical density; (v) modifying the illumination profile in (ii) by selecting the updated sample optical density as the optical density at the associated time to calculate an illumination for the selected depth value based on an attenuation of the selected incident illumination intensity and the updated sample optical density; (vi) exposing the algae sample to light intensity corresponding to the modified illumination profile; and characterizing at least one algae property for algae in the reference volume based on at least one measured algae property of the algae sample, optionally wherein steps (iv) and (v) are performed at least twice, preferably at regular intervals, for example, twice a day, daily, or every other day, and/or optionally wherein the culture is not diluted during the culturing period.

### Embodiment 3

The method of any one of Embodiments 1-2, wherein any one or any combination of the following are satisfied: the light intensity is directed toward the algae sample from a single side of the vessel, the product of the optical density and the sample depth is less than about 8.0 cm, less than about 6.0 cm, less than about 4.0 cm, less than about 3.0 cm, less than about 2.0 cm, or less than about 1 cm and/or culture absorbtion is at least 30%, at least 45%, or at least 50%, during the culture period.

### Embodiment 4

A method for comparing algae samples, comprising: (i) contriving a synthetic trajectory for a particle in a reference volume, the synthetic trajectory comprising at least a plurality of position values in the reference volume, the plurality of position values having associated continuous time values, the position values including at least a depth value relative to a light-incident surface of the reference volume; (ii) determining an illumination profile based on the particle trajectory by obtaining an illumination intensity corresponding to the plurality of position values and associated continuous time values; (iii) exposing a first algae sample in a first vessel to light intensity corresponding to the illumination profile, the first algae sample having a first sample depth, a first sample volume, and a first optical density, a product of the first optical density and the first sample depth being less than about 10.0 cm, the first sample volume being less than the reference volume; (iv) exposing a second algae sample in a second vessel to light intensity corresponding to the illumination profile, the second algae sample having a second sample depth, a second sample volume, and a second optical density, a product of the second optical density and the second sample depth being less than about 10.0 cm, the second sample volume being less than the reference volume, at least one environmental factor of the second vessel being different from a corresponding at least one environmental factor of the first vessel; (v) characterizing at least one algae property for the first algae sample; characterizing the at least one algae property for the second algae sample; and (vi) comparing the characterized at least one algae property for the first algae sample and the characterized at least one algae property for the second algae sample to determine the comparative effect of the at least one environmental factor, optionally wherein the at least one environmental factor that is different for the first algae sample and the second algae sample is a speed of mixing, a presence of a mixing structure, a presence of mixing jets, a CO₂ concentration, an O₂ concentration, a pH, the presence or concentration of a nutrient, an algae sample depth, a temperature, or a combination thereof.

### Embodiment 5

A method for comparing algae samples, comprising: (i) calculating a particle trajectory for a particle in a reference volume, the particle trajectory comprising at least a plurality of position values in the reference volume, the plurality of position values having associated times, the position values including at least a depth value relative to a surface of the reference volume; (ii) determining an illumination profile based on the particle trajectory by obtaining an illumination intensity corresponding to the plurality of position values and associated times; (iii) exposing a first algae sample comprising a first algal strain in a first vessel to light intensity corresponding to the illumination profile, the first algae sample having a first sample depth, a first sample volume, and a first optical density, a product of the first optical density and the first sample depth being less than about 10.0 cm, the first sample volume being less than the reference volume; (iv) exposing a second algae sample comprising a second algal strain in a second vessel to light intensity corresponding to the illumination profile, the second algae sample having a second sample depth, a second sample volume, and a second optical density, a product of the second optical density and the second sample depth being less than about 10.0 cm, the second sample volume being less than the reference volume; (v) characterizing at least one algae property for the first algae sample; characterizing the at least one algae property for the second algae sample; and (vi) comparing the characterized at least one algae property for the first algae sample and the characterized at least one algae property for the second algae sample to determine the difference in at least one algal property between the first algal strain and the second algal strain.

### Embodiment 6

The method of Embodiment 4 or Embodiment 5, wherein the first vessel and the second vessel are the same, the method further comprising: removing the first algae sample from the vessel after the exposure of the first algae sample to the illumination profile; and introducing the second algae sample into the vessel.

### Embodiment 7

The method of any one of Embodiments 4-6, wherein comparing the characterized at least one algae property for the first algae sample and the characterized at least one algae property for the second algae sample comprises: measuring one or more algae properties for the first algae sample; measuring the one or more algae properties for the second algae sample; characterizing the at least one algae property for the first algae sample based on the measured one or more algae properties for the first algae sample; and characterizing the at least one algae property for the second algae sample based on the measured one or more algae properties for the second algae sample.

### Embodiment 8

The method of any one of the previous embodiments, wherein one or more of the following are satisfied: the particle trajectory is a synthetic trajectory, such as an unreal trajectory and/or a discontinuous trajectory; determining an illumination profile comprises selecting a depth value and an associated time, selecting an intensity of illumination that is incident on a surface of the reference volume at the associated time, selecting an optical density at the associated time, and calculating an illumination for the selected depth value based on an attenuation of the selected incident illumination intensity and the selected optical density, preferably wherein the optical density α is defined by the equation αL=−log₁₀(I_{L}/I₀), where I₀ represents an intensity of light incident at a surface and I_{L} represents an intensity of light at distance L from the surface; the method further comprises determining a temperature profile at the associated times, the temperature profile being associated with at least one of the reference volume and the position values; characterizing at least one algae property for algae in the reference volume comprises withdrawing at least a portion of the algae sample and measuring a property of the withdrawn algae sample portion, optionally wherein the property is compared on a per OD, per ash free dry weight, per total organic carbon, per chlorophyll basis, or wherein measuring the property of the withdrawn sample portion includes calculating an algae growth rate, selecting an initial algae concentration for the reference volume, and characterizing the at least one algae property in the reference volume based on a relationship between the initial algae concentration for the reference volume, an algae concentration for the vessel or the withdrawn algae sample, and the calculated algae growth rate, optionally wherein selecting an initial algae concentration for the reference volume comprises determining an initial algae concentration based on a selected optical density for the reference volume; the sample volume of the algae sample, the first algae sample, or the second algae sample is less than about 50% of the reference volume, less than about 25% of the reference volume, preferably less than about 5% of the reference volume, and more preferably, less than about 1% of the reference volume; the at least one characterized algae property is biomass accumulation, ash free dry weight, total organic carbon, lipid accumulation, expression of one or more genes, activity of one or more enzymes, accumulation of one or more proteins, concentration of one ore more metabolites, growth rate, chlorophyll content, carotenoid content, oxygen evolution, non-photochemical quenching, or Fᵥ/Fₘ; and the sample volume of the algae sample, the first algae sample, or the second algae sample is less than about 5 L, e.g., less than about 2 L, less than about 1 L, less than about 500 mL, less than about 200 mL, or less than or equal to about 100 mL.

### Embodiment 9

A system for exposing algae samples to illumination, comprising: a vessel having a depth of about 10 cm or less and a cross-sectional area, the depth and the cross-sectional area corresponding to a vessel volume of less than about 5 liters; a plurality of light sources positioned so that emitted light is incident on a vessel surface having an area corresponding to the cross-sectional area; a plurality of lenses positioned to increase a percentage of emitted light that is incident on the vessel surface; a memory for storing at least a portion of an illumination profile; and a processor capable of controlling at least one power source for the plurality of light sources based on the stored at least a portion of an illumination profile, wherein the plurality of light sources and the plurality of lenses are positioned to be capable of delivering at least about 1000 μE/m²/sec PAR of illumination to the vessel surface, optionally wherein the plurality of light sources comprise at least one light emitting diode, and wherein the at least one power source optionally comprises a constant current controller.

### Embodiment 10

The system of Embodiment 9, further comprising: a thermoelectric heater in contact with a surface of the vessel; a memory for storing at least a portion of a temperature profile; a processor capable of controlling the thermoelectric heater based on the stored at least a portion of an temperature profile; optionally a manual or automated agitator; and/or optionally one or more of a thermometer, a thermocouple, a pH probe, and an aeration port.

### Embodiment 11

A method for selecting a strain of algal cells for biomass accumulation in photosynthetic culture, the method comprising: (a) co-culturing at least two different strains of algal cells; (b) exposing the algal cells to an illumination profile, wherein the illumination profile comprises light conditions that influence growth rate of at least one strain of algal cells; and (c) selecting a strain of algal cells for biomass accumulation based on a photosynthetic efficiency characteristic displayed by the selected strain of algal cells, optionally wherein steps (b) and (c) are repeated at least once more, optionally further comprising a step (d) of repeating steps (b) and (c) until one of the at least two different strains of algal cells becomes a dominant strain, and/or optionally further comprising a step (e) of isolating a culture of cells belonging to the dominant strain.

### Embodiment 12

The method of Embodiment 11, wherein one or more of the following is satisfied: the dominant strain comprises at least 50% of the cells in the growth chamber, for example at least 75% of the cells in the growth chamber; at least one strain of algal cells dies at a faster rate than another strain of algal cells; at least one strain of algal cells multiplies at a faster rate than another strain of algal cells; exposure time in step (b) increases after one or more iterations of steps (b) and (c); the light conditions that influence the growth rate of at least one strain of algal cells are photoinhibitory light conditions; the photosynthetic efficiency characteristic is diminished respiration, enhanced specific growth rate, diminished pigment concentration (e.g., diminished chlorophyll concentration), enhanced oxygen evolution, enhanced carbon fixation, or increased maximum tolerance of the chloroplast membrane to electrical potential; a cellular property is measured following exposure to light conditions in step (b), such as with a flow cytometer, for example which can remove at least some of the non-selected cells from the culture; a physiological or environmental parameter is measured during step (b) and/or (c), for example by sampling off-gas, which physiological or environmental parameter can be selected from the group consisting of temperature, pH, carbon concentration, oxygen concentration, fluorescence, and combinations thereof; the biomass accumulation comprises at least one lipid, protein, and/or polysaccharide; the at least two strains of algal cells are cultured in a vessel that results in no more than 30% loss of light intensity across the optical depth of the vessel; no more than 10% of light intensity is lost across the optical depth of the vessel; and the at least two strains of algal cells are cultured in a vessel that results in more than 30% loss of light intensity across the optical depth of the vessel.

### Embodiment 13

A system for selecting a strain of algal cells for biomass accumulation, the system comprising: (a) a growth chamber connected to a selection chamber, wherein at least a portion of each of the growth and selection chamber are substantially translucent; (b) a plurality of light sources positioned to illuminate the substantially translucent portions of the growth and selection chambers; (c) a plurality of lenses positioned to increase emitted light that is incident on the substantially translucent portions of the growth and selection chambers; and optionally (d) a means for controlling temperature in the growth chamber and/or the selection chamber.

### Embodiment 14

The system of Embodiment 13, where in one or more of the following are satisfied: the system further comprises a means for controlling temperature in the growth chamber and a separate means for controlling temperature in the selection chamber, the system further comprises a flow cytometer, which can optionally comprise a hydraulic connection between the growth chamber and the selection chamber; the growth chamber has an optical depth at least about ten times larger than the selection chamber, and the selection chamber comprises a port for sampling off-gas.

### Embodiment 15

A method for determining an effect of a physiological or environmental parameter on algal photosynthetic efficiency, the method comprising: (a) calculating a synthetic trajectory for a particle in a reference volume, the synthetic trajectory comprising at least a plurality of position values in the reference volume, the plurality of position values having continuous associated time values, the position values including at least a depth value relative to a light-incident surface of the reference volume; (b) forming an illumination profile for the synthetic trajectory by determining light intensity corresponding to each of the plurality of position values and associated times; (c) exposing an algae sample to light intensity corresponding to the illumination profile formed in step (b), and (d) characterizing at least one effect of the physiological or environmental parameter on algal photosynthetic efficiency of the algae sample.

### Embodiment 16

The method of Embodiment 15, wherein one or more of the following is satisfied: the synthetic trajectory is an unreal trajectory; the position values are discontinuous; the synthetic trajectory defines an illumination profile selected from the group consisting of an illumination profile with periodic movements between dark and light, an illumination profile with quasiperiodic movements between dark and light, an illumination profile with irregular movements between light and dark, an illumination profile in which at least a portion of the illumination profile includes a discrete, step-wise escalation or de-escalation of light intensities, and a combination thereof; the at least one effect of the physiological or environmental parameter is an effect on specific growth rate; and the at least one effect of the physiological or environmental parameter is an effect on simulated density (K value) of the culture.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Determining a Specific Growth Rate for Light-Adapted Cultures

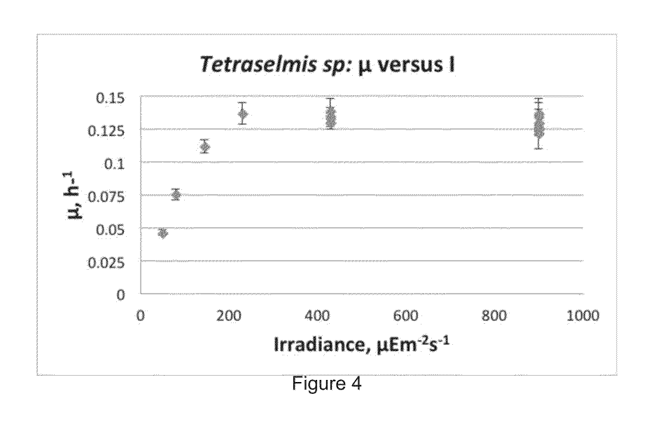

In order to explore the effect that a cell's history can have on the cell's instantaneous photosynthetic properties, dilute cultures of Tetraselmis sp. 2286 cells were grown in standard culture media, and individual cultures were exposed to ˜50, ˜100, ˜150, ˜250, ˜450, and ˜900 μE/m²/sec irradiance, respectively. During the course of the experiment, all cultures were diluted daily in order to maintain optically dilute suspensions and all cultures were maintained within a range of pH ˜6.8˜7.2 by sparging all cultures with a ˜99.25%/˜0.75% air/CO₂ mixture through an air stone. Ambient room temperature conditions were adjusted as necessary to maintain the cultures at a range between about 28-30° C. during the course of the experiment. Specific growth rate, calculated according to the equation Ln(biomass (t₂)/biomass (t₁))/(t₂−t₁), where t₂ is a time point arbitrarily later than t₁, was calculated for each individual culture and plotted in FIG. 4. As can be seen in FIG. 4, specific growth rate was observed to increase rapidly in proportion to irradiance between ˜0 and ˜250 μE/m²/sec, but did not continue to increase beyond ˜250 μE/m²/sec. In other words, ˜0.13 per hour, achieved at an irradiance of about 250 μE/m²/sec, is the maximum specific growth rate (μₘₐₓ) observed with Tetraselmis sp. 2286 cells with this particular light adaptation history.

To illustrate the path dependency of the μₘₐₓ, cultures of Tetraselmis were first adapted to different light conditions before subjecting them to similar experimental conditions to those described immediately above. To summarize briefly, individual dilute cultures of Tetraselmis sp. 2286 were grown for an extended period of time at ˜50, ˜430, and ˜900 μE/m²/sec respectively. Individual dense cultures of Tetraselmis sp. 2286 were also grown at ˜900 μE/m²/sec for an extended period of time.

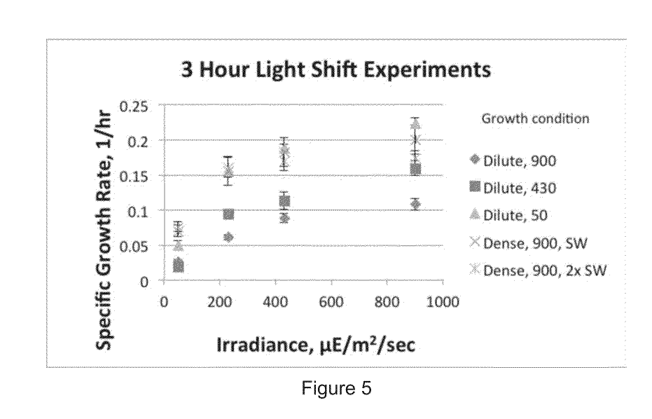

After an extended period of growth in the light conditions described above, each of these cultures were diluted to equal optical densities and divided four ways. One division from each acclimation condition was shifted to growth for about three hours at ˜50, ˜250, ˜450, or ˜900 μE/m² sec respectively. Specific growth rates, calculated as above, were computed for each culture, and plotted in FIG. 5. As can be seen, each light acclimation condition gave rise to its own unique μₘₐₓ value following the light intensity shift, despite the fact that all cultures involved the same cell type. It is also noteworthy that the cells that had been acclimated to low light conditions achieved much higher specific growth rates at any given level of post-shift irradiance.

### Example 2

### Effect of Light Sequence (Mixing) on Growth Simulation

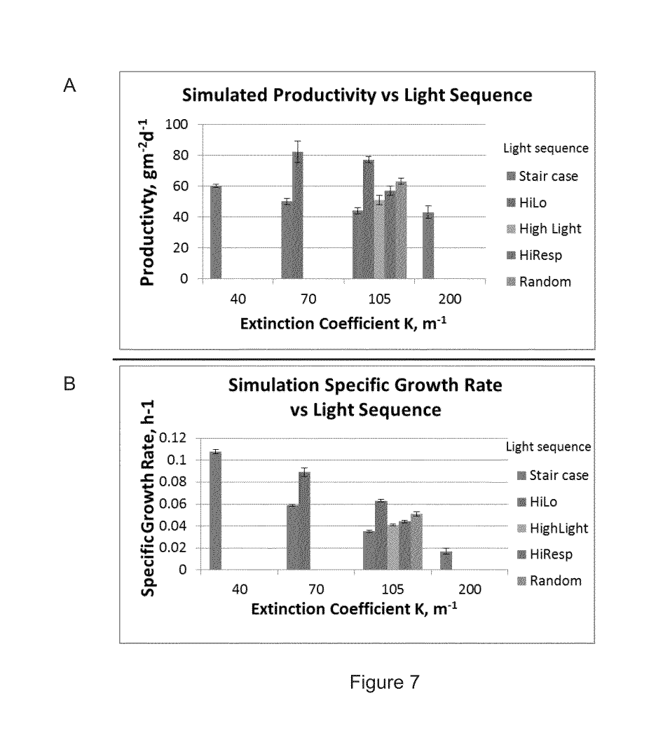

In order to further characterize the effect that the experience of light can have on the subsequent response of a cell to new light conditions, cultures of Tetraselmis cells in cultures with one of four distinct extinction coefficients (˜40, ˜70, ˜105, or ˜200 m⁻¹) were exposed to various patterns of light exposure. Following patterned light exposure, biomass productivity was calculated using simulation parameters measured in dilute cultures (FIG. 7A) and/or specific growth rates were measured for the dilute culture (FIG. 7B).

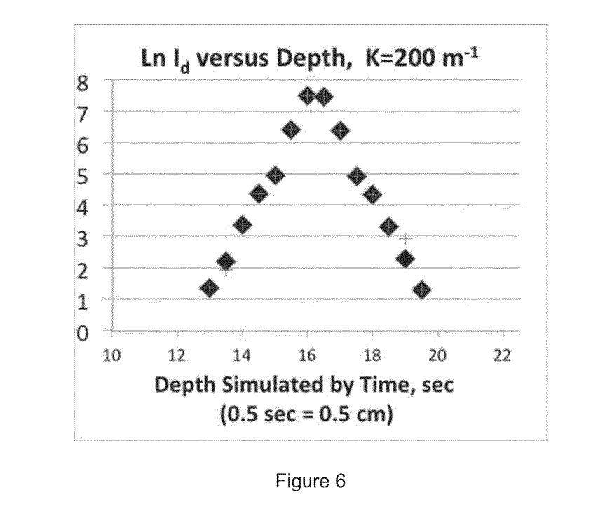

As summarized in FIG. 7A, cultures with extinction coefficients of ˜40, ˜70, ˜105, and ˜200 m⁻¹ were exposed to the “stair case” light pattern exemplified in FIG. 6. Another set of cultures (“HiLo”) with extinction coefficients of ˜70 and ˜105 m⁻¹ were exposed to an alternating series of “high” and “low” light conditions; the “HiLo” conditions involved exposing cells to the highest light value (˜2000 μE/m²/sec), followed by the lowest (˜0 μE/m²/sec), followed by the second highest, followed by the second lowest, etc. Three more cultures, all with an extinction coefficient of ˜105 m⁻¹, were exposed to “High” light (six consecutive seconds total of light from ˜700 μE/m²/sec to ˜2000 μE/m²/sec and back down to ˜700 μE/m²/sec, followed by ˜28 seconds of light alternating, second by second, between the third highest light level, (˜400 μE/m²/sec) and the lowest light level (˜0 μE/m²/sec), followed by the fourth highest and the second lowest, etc., to induce photoinhibition, continuous HiResp conditions (about eighteen seconds of low light, from ˜30 μE/m²/sec to about zero and about zero to ˜30 μE/m²/sec, with about sixteen seconds of light alternating from the tenth lowest (˜51 μE/m²/sec) to the highest (˜2000 μE/m²/sec), the eleventh lowest to the second highest, etc., to induce respiration, or random light conditions, respectively. Following these light exposures, productivity of the cultures was assessed, as above by calculating the weight (grams) of ash-free dry mass generated per day of culture and, in addition, measuring specific growth rates.

### Example 3

### Growth of Cultures in Partially Covered Ponds

It has previously been found that when an open pond is partially covered, blocking out some of the incident light, the way in which the cover is placed determines the reduction observed in biomass productivity. A pond that is covered contiguously over ˜75% of its area produces ˜25% as much biomass as an uncovered pond, as would be expected. That is to say, the photosynthetic efficiency (PE) on light incident to the culture suspension is the same as for a totally uncovered pond. A pond that is entirely covered by a shade cloth blocking ˜70% of the incident light produces about 60% as much as an uncovered pond, which is about twice as much as would be expected if the remaining light incident on the culture were used at the same efficiency. The PE of the shade-cloth covered pond is thus higher, twice as high, because the incident intensity was lower. However, this result is also expected, as a shade cloth reduces incident light intensity and, thus, ameliorates the light saturation effect.

However, if a pond is covered ˜75% with opaque slats (e.g., ˜22.5 cm strips of thick, white, polypropylene plastic) spaced ˜7.5 cm apart (thus allowing the culture to receive only ˜25% of the incident light intensity), this slat-covered pond will produce somewhat over 50% of the productivity of the uncovered pond, not ˜25% as expected. If the horizontal mixing speed is about 15 cm/sec, the algal suspension (though not the individual cells) will cycle in and out of the light on an interval of ˜0.5 sec and ˜1.5 sec. respectively. That is, the slat-covered pond's PE will be substantially higher, about twice that of the uncovered pond. The incident light intensity received by the cultures in the slat-covered pond, however, is not decreased over the open area and, thus, light saturation cannot explain this increase in relative productivity (per unit area of light received).

The most logical explanation for this phenomenon is an amelioration of light inhibition. It appears that cell suspensions subjected to the high light continuously (as in the completely open pond), or for longer periods than a few seconds (as in the ˜75% contiguously covered ponds) are inhibited in their photosynthetic efficiency. This inhibition could be relieved to some extent by exposing them only intermittently to the high light at relatively short cycle times (e.g. less than one second in the light). These cycling times are much too long to be attributed to the flashing light effect first describe by Kok (1953), in which cells are exposed to the light for a few milliseconds and then are kept in the dark for five to ten times longer periods. A great deal of literature over the years has suggested that longer periodicities can increase photosynthetic conversion efficiencies (e.g. Laws et al., 1986, Laws and Berning, 1991). Thus, further investigation of this phenomenon was warranted.

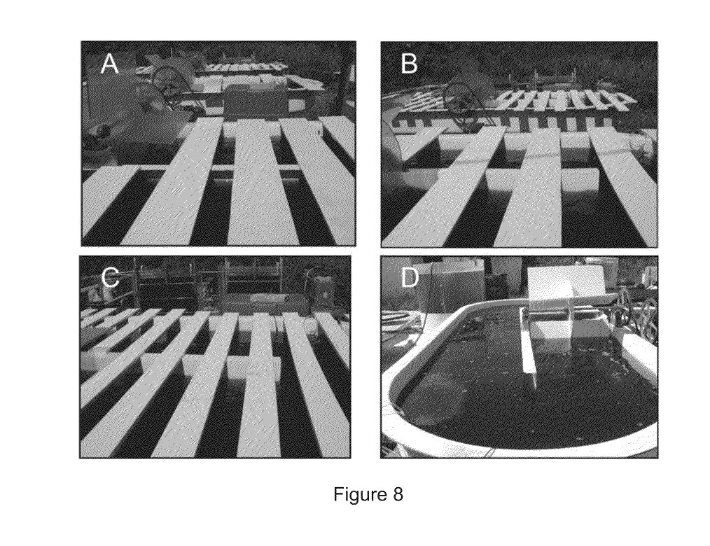

In a first experiment, 45% of the surface of one pond was covered by slats ˜15 cm wide, placed about ˜15 cm apart (FIG. 8B). The total coverage was not ˜50% because the slats could not be arranged without a remainder near the edge of the pond. The surface of another pond (control) was open (FIG. 8D). The biomass production from a pond with ˜55% of the light input was ˜80±2% of that from an uncovered pond. The corresponding PE was ˜46±2% greater.

In a subsequent experiment, the ˜15 cm slats were placed ˜7.5 cm apart, leaving only ˜33% of the pond open (FIG. 8A). Now the biomass production was two thirds of the open pond and the PE doubled. To determine whether the time in the light or the time out of the light was more important by the ˜15 cm slats, ˜7.5 cm apart arrangement was compared to ponds for which ˜15 cm slats were placed ˜15 cm apart. In the other case, ˜7.5 cm wide slats were placed ˜7.5 cm apart (FIG. 8C). In other words, the three ponds provided, among them, two treatments with the suspensions exposed to the light for ˜7.5 cm (about 0.5 sec), but with different times in the dark (˜1 sec versus ˜0.5 sec), and two treatments with exposure to the dark for ˜15 cm (˜1 sec), but different times in the light. Results are given in Table 2. The length of time in the light or dark did not matter with about 50% coverage. With only ˜52 or ˜55% as much light input, the productivity was still ˜80% as much as an uncovered pond. PE was thus about 50% greater than the uncovered control. Comparing the ˜33% open case to the 50+% open cases, that is adding back roughly 50% more light (at high intensity), increased productivity only about 15%.

The analysis is blurred to some extent by the effects of diffuse light. The photosynthetic efficiencies for intermittently covered ponds were overestimated because the light intensity under the slats was not zero. It was estimated to about 5-8% of the unobstructed intensity, based on a few individual measurements. The amount of light under the slats was minimized by having the slats as close to the pond surface as possible, by operating at greater depth. However, this was limited by the need for freeboard to hold rainwater. Therefore diffuse light still hit the pond surface under the slats.

With ˜10-15% more light impinging on the pond than calculated based on the open area, instead of expecting ˜50% of the productivity compared to a totally open pond, one should expect ˜60-65% as much, based strictly on total light input. The results, however, showed considerably more productivity, at about 80% of the open ponds, a significant effect. The amount of diffuse light also depended on the width of the slats. The comparison of wide slats to narrow slats shown in Table 2 gives some idea of the magnitude of the error due to diffuse light under the slats. The light intensity under wide slats was less than that under narrow slats. In the two trials, the pond covered with narrow slats was ˜5% and ˜10% more productive than the one covered with wide slats.

TABLE 2
Biomass from 3 m² outdoor ponds in Florida.

| Uncovered surface | Relative biomass productivity | Relative PE |
|---|---|---|
| ~100% | ~100% | ~100% |
| ~52% | ~80% | ~160% |
| ~33% | ~70% | ~220% |

Although the present invention has been described in terms of specific embodiments, it is not so limited. Suitable alterations/modifications for operation under specific conditions should be apparent to those skilled in the art. It is therefore intended that the following claims be interpreted as covering all such alterations/modifications as fall within the true spirit/scope of the invention.

What is claimed is:

1. A method for selecting a strain of algal cells for biomass accumulation in photosynthetic culture, the method comprising:
(a) co-culturing at least two different strains of algal cells;
(b) exposing the algal cells to an illumination profile, wherein the illumination profile comprises light conditions that influence growth rate of at least one strain of algal cells; and
(c) selecting a strain of algal cells for biomass accumulation based on a photosynthetic efficiency characteristic displayed by the selected strain of algal cells.

2. The method of claim 1, wherein steps (b) and (c) are repeated at least once more.

3. The method of claim 1, further comprising the step:
(d) repeating steps (b) and (c) until one of the at least two different strains of algal cells becomes a dominant strain.

4. The method of claim 3, further comprising the step:
(e) isolating a culture of cells belonging to the dominant strain.

5. The method of claim 3, wherein the dominant strain comprises at least 50% of the cells in the growth chamber.

6. The method of claim 5, wherein the dominant strain comprises at least 75% of the cells in the growth chamber.

7. The method of claim 1, wherein at least one strain of algal cells dies at a faster rate than another strain of algal cells.

8. The method of claim 1, wherein at least one strain of algal cells multiplies at a faster rate than another strain of algal cells.

9. The method of claim 2, wherein exposure time in step (b) increases after one or more iterations of steps (b) and (c).

10. The method of claim 1, wherein the light conditions that influence the growth rate of at least one strain of algal cells are photoinhibitory light conditions.

11. The method of claim 1, wherein the photosynthetic efficiency characteristic is diminished respiration, enhanced specific growth rate, diminished pigment concentration, enhanced oxygen evolution, enhanced carbon fixation, or increased maximum tolerance of the chloroplast membrane to electrical potential.

12. The method of claim 11, wherein the diminished pigment concentration is diminished chlorophyll concentration.

13. The method of claim 1, wherein a cellular property is measured following exposure to light conditions in step (b).

14. The method of claim 13, wherein the cellular property is measured with a flow cytometer.

15. The method of claim 14, wherein the flow cytometer removes at least some of the non-selected cells from the culture.

16. The method of claim 1, wherein a physiological or environmental parameter is measured during step (b) and/or (c).

17. The method of claim 16, wherein the physiological or environmental parameter is selected from the group consisting of temperature, pH, carbon concentration, oxygen concentration, and fluorescence.

18. The method of claim 16, wherein the physiological or environmental parameter is measured by sampling off-gas.

19. The method of claim 1, wherein the biomass accumulation comprises at least one lipid and/or polysaccharide.

20. The method of claim 1, wherein the at least two strains of algal cells are cultured in a vessel that results in no more than 30% loss of light intensity across the optical depth of the vessel.

21. The method of claim 20, wherein no more than 10% of light intensity is lost across the optical depth of the vessel.

22. The method of claim 1, wherein the at least two strains of algal cells are cultured in a vessel that results in more than 30% loss of light intensity across the optical depth of the vessel.

23. A system for selecting a strain of algal cells for biomass accumulation, the system comprising:
(a) a growth chamber connected to a selection chamber, wherein at least a portion of each of the growth and selection chamber are substantially translucent;
(b) a plurality of light sources positioned to illuminate the substantially translucent portions of the growth and selection chambers; and
(c) a plurality of lenses positioned to increase emitted light that is incident on the substantially translucent portions of the growth and selection chambers.

24. A method for determining an effect of a physiological or environmental parameter on algal photosynthetic efficiency, the method comprising:
(a) contriving a synthetic trajectory for a particle in a reference volume, the synthetic trajectory comprising at least a plurality of position values in the reference volume, the plurality of position values having continuous associated time values, the position values including at least a depth value relative to a light-incident surface of the reference volume;
(b) forming an illumination profile for the synthetic trajectory by determining light intensity corresponding to each of the plurality of position values and associated times;
(c) exposing an algae sample to light intensity corresponding to the illumination profile formed in step (b), and
(d) characterizing at least one effect of the physiological or environmental parameter on algal photosynthetic efficiency of the algae sample.

25. The method of claim 24, wherein the synthetic trajectory is an unreal trajectory.

26. The method of claim 25, wherein the position values are discontinuous.

27. The method of claim 25, wherein the synthetic trajectory defines an illumination profile selected from the group consisting of: an illumination profile with periodic movements between dark and light; an illumination profile with quasiperiodic movements between dark and light; an illumination profile with irregular movements between light and dark; and an illumination profile in which at least a portion of the illumination profile includes a discrete, step-wise escalation or de-escalation of light intensities.

28. The method of claim 25, wherein the at least one effect of the physiological or environmental parameter is an effect on specific growth rate.

29. The method of claim 25, wherein the at least one effect of the physiological or environmental parameter is an effect on simulated density (K value) of the culture.
